SEQUENCE LISTING (16 sequences)

SEQ ID NO: 1 - protein, 15 aa, synthetic construct
DGENDDPRPPNPPKC

SEQ ID NO: 2 - protein, 107 aa, synthetic construct
DIQLTQTTSSLSASLGDRVTISCSASQGISNYLSWYQQKPDGTFKLLIYYTSTLHSGVPSRFSGSGSGTDYSLTISNLEPEDIATYYCQQYSNLPWTFGGGTRLEIK

SEQ ID NO: 3 - protein, 122 aa, synthetic construct
EVQLQQSGPELVKPGASVKISCKASGYSFTGYYMHWVKQSHVKSLEWIGRINPYTGATTYNQIFKDKATLTLDKSSSTAYMELHSLTSEDSAIFYCARYYYGNNYNVYLDYWGQGTTLTVSS

SEQ ID NO: 4 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - DNA, 451 bp, synthetic construct
  source 1..451: mol_type = other DNA
gaggtccagctgcagcagtctggacctgagctggtgaagcctggggcttcagtgaagatatcctgcaaggcttctggttactcattcactggctactacatgcactgggtgaagcaaagccatgtaaagagccttgagtggattggacgtattaatccttacactggtgctactacctacaaccagattttcaaggacaaggccaccttgactttagataagtcctccagcacagcctacatggagctccacagcctgacatcagaggactctgcaatcttttactgtgcaagatattactacggtaataattacaatgtctacttggactactggggccaaggcaccactctcacagtctcctcagccaaaacaacagccccatcggtctatccactggcccctgtgtgtggagatacaactggctcctcggtgactctaggatgcctgg

SEQ ID NO: 6 - protein, 5 aa, synthetic construct
GYYMH

SEQ ID NO: 7 - protein, 17 aa, synthetic construct
RINPYTGATTYNQIFKD

SEQ ID NO: 8 - protein, 13 aa, synthetic construct
YYYGNNYNVYLDY

SEQ ID NO: 9 - DNA, 335 bp, synthetic construct
  source 1..335: mol_type = other DNA
agttgacacagactacatcctccctgtctgcctctctgggagacagagtcaccatcagttgcagtgcaagtcagggcattagtaattatttaagctggtatcagcagaaaccagatggaacttttaaactcctgatctattacacatcaactttacactcaggagtcccatcaaggttcagtggcagtgggtctgggacagattattctctcaccatcagcaacctggaacctgaagatattgccacttactattgtcagcaatatagtaaccttccgtggacgttcggtggaggcaccagactggaaatcaaacgggctgatgctgcaccaact

SEQ ID NO: 10 - protein, 11 aa, synthetic construct
SASQGISNYLS

SEQ ID NO: 11 - protein, 7 aa, synthetic construct
YTSTLHS

SEQ ID NO: 12 - protein, 9 aa, synthetic construct
QQYSNLPWT

SEQ ID NO: 13 - protein, 540 aa, synthetic construct
MGWSWIFLFLLSGTAGVLSEVQLQQSGPELVKPGASVKISCKASGYSFTGYYMHWVKQSHVKSLEWIGRINPYTGATTYNQIFKDKATLTLDKSSSTAYMELHSLTSEDSAIFYCARYYYGNNYNVYLDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGLDLDDVCAEAQDGELDGLWTTITIFISLFLLSVCYSASVTLFKVKWIFSSVVELKQTISPDYRNMIGQGA

SEQ ID NO: 14 - DNA, 1623 bp, synthetic construct
  source 1..1623: mol_type = other DNA
atgggatggagctggatctttctctttctcctgtcaggaactgcaggtgtcctctctgaggtccagctgcagcagtctggacctgagctggtgaagcctggggcttcagtgaagatatcctgcaaggcttctggttactcattcactggctactacatgcactgggtgaagcaaagccatgtaaagagccttgagtggattggacgtattaatccttacactggtgctactacctacaaccagattttcaaggacaaggccaccttgactttagataagtcctccagcacagcctacatggagctccacagcctgacatcagaggactctgcaatcttttactgtgcaagatattactacggtaataattacaatgtctacttggactactggggccaaggcaccactctcacagtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccggggctagacctggatgatgtctgtgctgaggcccaggacggggagctggacggcctctggacgaccatcaccatcttcatcagcctcttcctgctcagcgtgtgctacagcgcctctgtcacactcttcaaggtaaagtggatcttctcctctgtggtggagctgaagcagacgatctcccctgactacagaaacatgattgggcagggagcctag

SEQ ID NO: 15 - protein, 234 aa, synthetic construct
MMSSAQFLGLLLLCFQGTRCDIQLTQTTSSLSASLGDRVTISCSASQGISNYLSWYQQKPDGTFKLLIYYTSTLHSGVPSRFSGSGSGTDYSLTISNLEPEDIATYYCQQYSNLPWTFGGGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - DNA, 705 bp, synthetic construct
  source 1..705: mol_type = other DNA
atgatgtcctctgctcagttccttggtctcctgttgctctgttttcaaggtaccagatgtgatatccagttgacacagactacatcctccctgtctgcctctctgggagacagagtcaccatcagttgcagtgcaagtcagggcattagtaattatttaagctggtatcagcagaaaccagatggaacttttaaactcctgatctattacacatcaactttacactcaggagtcccatcaaggttcagtggcagtgggtctgggacagattattctctcaccatcagcaacctggaacctgaagatattgccacttactattgtcagcaatatagtaaccttccgtggacgttcggtggaggcaccagactggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

(19) United States
(12) Patent Application Publication
(10) Pub. No.: US 2025/0340666 A1
(43) Pub. Date: Nov. 6, 2025

(54) DEVELOPMENT OF A NOVEL THERAPEUTIC CD99 ANTIBODY TO TREAT AGGRESSIVE SOLID TUMORS IN CHILDREN

(71) Applicant: The Regents of the University of Colorado, A Body Corporate, Denver, CO (US)

(72) Inventors: Sujatha Venkataraman, Aurora, CO (US); Rajeev Vibhakar, Aurora, CO (US)

(21) Appl. No.: 18/870,190

(22) PCT Filed: May 31, 2023
(86) PCT No.: PCT/US2023/067715

§ 371 Date: Nov. 27, 2024

Related U.S. Application Data
(60) Provisional application No. 63/347,806, filed on Jun. 1, 2022.
(60) Provisional application No. 63/348,443, filed on Jun. 2, 2022.
(60) Provisional application No. 63/402,429, filed on Aug. 30, 2022.

(51) Int. Cl. C07K 16/28; A61K 39/00; A61P 35/00

(52) U.S. Cl. CPC C07K 16/2896; A61P 35/00; A61K 2039/505

(57) ABSTRACT

Methods, compositions, and systems for treating various cancers are disclosed. The disclosed compositions may include a poly peptide with affinity for a CD99 cell surface protein. Disclosed polypeptides may comprise a sequence selected from GYYMH, RINPYTGATTYNQIFKD, YYYGNNYNVYLDY, SASQGISNYLS, YTSTLHIS, and QQYSNLPWT, and may include mouse, human, or humanized peptide sequences. In many embodiments, the polypeptides may be immunoglobulins, for example IgG3 or IgG4. The disclosed polypeptides may be administered to a subject having a cancer cell with elevated expression of CD99. In some embodiments, the subject may be suffering from cancer, including diffuse intrinsic pontine glioma (DIPG). Ewing Sarcoma, acute myeloid leukemia (AML), ependymoma, or neuroblastoma. Treatment methods include administering the disclosed polypeptides to a subject that may also be treated with radiation. Disclosed herein are systems for treating one or more cancers. The systems may comprise a radiation source, for example a medical fractionated radiation source.

### CROSS-REFERENCE TO RELATED APPLICATIONS

This application claims benefit of priority pursuant to 35 U.S.C. § 119(e) of U.S. provisional patent application Nos. 63/347,806 filed Jun. 1, 2022: 63/348,443 filed Jun. 2, 2022; and 63/402,429 filed Aug. 30, 2022, all entitled “DEVELOPMENT OF A NOVEL THERAPEUTIC CD99 ANTIBODY TO TREAT AGGRESSIVE SOLID TUMORS IN CHILDREN” which are hereby incorporated by reference in their entirety.

### FIELD

The present disclosure is directed to novel therapeutic compounds, therapies, and systems for identifying, assessing, and treating various cancers.

### SEQUENCE LISTING

The instant application contains a Sequence Listing which has been submitted electronically in xml format and is hereby incorporated by reference in its entirety. Said xml file, created on 16 May 2023, is named P290694_CH0082H.xml and is 19,327 bytes in size.

### BACKGROUND

Brain tumors are the leading cause of cancer-related deaths in children [1]. One of the most devastating types of brain tumors is DIPG (Diffuse intrinsic pontine glioma) [2, 3]. Treatment options for DIPG are limited as responses to radiation are only temporary. Chemotherapy is largely ineffective and surgical resection is not possible due to the tumor's location in the pons, a region of the brain responsible for multiple vital functions like heartbeat and respiration [4, 5]. The 5-year survival rate has held steady at 0% since 1950 as conventional therapies fail to improve local control or survival of this tumor type. Thus, there is an urgent unmet need to identify novel targeted therapies for treating this patient population.

### BRIEF SUMMARY

Disclosed herein are antibodies with affinity to the cell surface protein CD99, comprising: a Heavy Chain CDR1 of SEQ ID NO: 6, a Heavy Chain CDR2 of SEQ ID NO: 7, a Heavy Chain CDR3 of SEQ ID NO: 8, a Light Chain CDR1 of SEQ ID NO: 10, a Light Chain CDR2 of SEQ ID NO: 11, and a Light Chain CDR3 of SEQ ID NO: 12. In various embodiments, the CD99 antibody comprises a Variable Light Chain of SEQ ID NO: 2 and a Variable Heavy Chain of SEQ ID NO: 3, and may further comprise an IgG4 Fc, for example of SEQ ID NO: 4. The anti-CD99 antibody, in many embodiments, may be useful for treatment of various cancers, including acute myeloid leukemia (AML), neuroblastoma, ependymoma, DIPG, or Ewing Sarcoma and/or may be included in a therapeutic composition for treatment thereof. In some embodiments, the disclosed anti-CD99 antibody may be used in methods of inducing apoptosis in. reducing growth and/or inhibiting growth of a cancer cell, wherein the method may comprise steps of contacting a CD99 protein on a surface of the cancer cell with the anti-CD99 antibodies. In various embodiments, treatment of a subject or patient with the disclosed anti-CD99 antibody may be combined with radiation treatment of the same cancer tissue or tumor.

Also disclosed are methods of treating various disease, for example cancer, such as acute myeloid leukemia (AML), neuroblastoma. ependymoma. DIPG, or Ewing Sarcoma in a subject in need thereof, wherein the methods comprise steps including administering to the subject a therapeutic amount of an antibody having affinity for an epitope of CD99. In many embodiments, the cancer is a solid tumor cancer. In many embodiments, the antibody binds an epitope of CD99 having a sequence of SEQ ID NO: 1. In some embodiments, the antibody may be an IgG4 antibody. In various embodiments, the antibody may be chimeric or humanized and/or may comprise a Heavy Chain CDR1 of SEQ ID NO: 6, a Heavy Chain CDR2 of SEQ ID NO: 7, a Heavy Chain CDR3 of SEQ ID NO: 8, a Light Chain CDR1 of SEQ ID NO: 10, a Light Chain CDR2 of SEQ ID NO: 11, and a Light Chain CDR3 of SEQ ID NO: 12. In some embodiments, the Variable Light Chain is of SEQ ID NO: 2 and the Variable Heavy Chain is of SEQ ID NO: 3.

The CDR and variable chain sequences disclosed herein may be less than 100% identical to the sequences of the SEQ ID NOs provided herein. in some embodiments, the identity is less than 99%. 98%. 97%. 96%. 95%, 94%. 93%, 92%, 91%, 90%. 85%. 80%. 75% or 70%. and greater than about 65%, 70%, 75%, 80%. 85%, 90%, 91%, 92%, 93%, 94%, 95%, 96%. 97%, 98%, or 99%. In many embodiments, the CDR sequences are greater than 95% identical, and the variable sequences are greater than about 80% identical. In many embodiments, the presently claimed variable region sequences and framework sequences may include one or more conservative substitutions.

Also disclosed are methods for treating a solid tumor, comprising steps of directing radiation energy toward the solid tumor and contacting at least one cell within the solid tumor with an anti-CD99 antibody. thereby treating the solid tumor.

The disclosed methods and compositions do not affect cell proliferation or induce cell death in normal cells.

### BRIEF DESCRIPTION OF THE SEVERAL VIEWS OF THE DRAWINGS

FIG. 1 shows expression related to an embodiment of the present disclosure showing that CD99 is elevated in DIPG patient tumors and cell lines. Panel A is a volcano plot of differentially expressed genes in GBM01+H3K27M cells that shows a significant increase in CD99 mRNA (arrow) against the control (WT H3) cells. Panel B shows the expression of CD99 mRNA in K27M+ DIPG patient samples ('R2: Genomics Analysis and Visualization Platform (http://r2.amc.nl). Panel C is a representative immuno-histochemical staining of CD99 showing a prominent expression of CD99 in H3K37M+-DIPG (SF 5606) and no expression in the normal pons. Panel D shows that H3K27M+DIPG cells express high levels of surface CD99 compared to fetal normal human astrocytes (NHA).

FIG. 2 shows various experimental results related to an embodiment of the present disclosure. Analysis of a western blot showing high CD99 protein expression in a cohort of DIPG patient tumor tissues (Panel A) and in DIPG cell lines (Panel B) compared to normal pons (Normal pons lanes have higher protein loading to detect CD99). In Panel B, two isoforms 28 kDa (short form) and 32 kDa (long. active form) of CD99 and alpha-tubulin housekeeping gene are shown. (Pane C) Flow cytometry plots showing the high expression of CD99 on DIPG tumor cells.

FIG. 3 shows the correlation of CD99 cell surface expression with H3K27M mutation related to an embodiment of the present disclosure. Flow staining of BT245 (+H3K27M) and H3K27M-knockout BT245 (H3K27M-KO) cells showed high expression of CD99 in mutant H3K27M positive cells, which is substantially lost (˜95%) in the H3K27M-KO cells.

FIG. 4 shows genetic Knockdown of CD99 leads to a decrease in DIPG cell tumor growth related to an embodiment of the present disclosure. Two different shRNAs targeting CD99 were used (503 and 644). Shnull=control non-targeting control.

FIG. 5 shows the genetic knockdown of CD99 (ShRNA) delayed tumor initiation in vivo related to an embodiment of the present disclosure, where BT245-LUC cells expressing high CD99 (shnull) and low CD99 (KD of CD99 by shRNA) were implanted in mouse pons. Panel A is a representative IVIS imaging of tumor establishment is shown in both cohorts of mice (Shnull and shCD99). Panel B presents IVIS signal intensities measured at different weeks after tumor implantation. And Panel C shows a quantitative analysis of GFAP in the tumor tissue collected at the endpoint.

FIG. 6 CRISPR KO of CD99 from DIPG cells inhibited tumor establishment. Representative IVIS images of the animals implanted with DIPG control (left) and CD99 deleted tumor cells (right).

FIG. 7 is a depiction of the full-length CD99 protein and the commercially available antibody epitopes. (Pasello M, Manara MC, Scotlandi K. CD99 at the crossroads of physiology and pathology. J Cell Commun Signal. 2018 March; 12 (1): 55-68.) The presently disclosed novel anti-CD99 antibody (10D1) sequence is shown in yellow.

FIG. 8 presents experimental results showing that the 10D1-CD99 antibody effectively blocked CD99 expression on DIPG cells even at a low concentration (10 ug) of the antibody as measured by flow cytometry using 0662 CD99 antibody.

FIG. 9 shows that the 10D1-CD99 antibody is specific in detecting the cell surface CD99 protein. DIPG tumor tissues were stained with different commercially available CD99antibodies along with 10D1-CD99 antibodies and IHC images are shown from the staining.

FIG. 10 illustrates an aspect of the subject matter in accordance with one embodiment of blocking CD99 using 10D1 antibody decreased tumor cell growth in a patient primary tumor, MAF-001 (Panel A). Panel B—shows that no toxicity was seen when the normal NHA cells were treated with 20 ug of the CD99 antibody. Panel C shows that treatment of DIPG04 cells with 10D1 antibody led to an increase in tumor cell death.

FIG. 11 shows the enhanced anti-tumor efficacy of the 10D1 CD99 antibody against DIPG. Panel A shows DIPG tumor burden monitored by in vivo bioluminescence imaging (BLI). DIPG luciferase-expressing (blue) cells were implanted in the mouse pons, and after tumor establishment (day 9), mice were treated with 10DI antibody at 1 mg/kg of mice and 8 mg/kg by tail vein injection. Complete clearance of DIPG tumor after four doses of 8 mg/kg of the antibody are seen. Panel B shows the flux intensities of imaging, and the changes in the tumor burden with the treatment are shown in the adjacent graph. Panel C is a Kaplan-Meier survival analysis showing an increase in animal survival with the 10D1 antibody treatment.

FIG. 12 shows results from loco-regional delivery of the 10D1 antibody that is efficient in clearing DIPG tumors in mice, even at a low dose. Representative images of DIPG tumor cells after treatment with either control IgG4 or 10D1-CD99 antibody delivered (Panel A) in the lateral ventricle in DIPG (BT245-LUC) or (Panel B) at the tumor site, pons in DIPG (UPN1587-LUC) xenograft mouse models. For both these antibody delivery models, a single dose of 1 mg/kg of the antibody or the control, IgG4, was delivered.

FIG. 13 shows radiosensitization of DIPG cells to 10D1 antibody. Panels A-C show an increase in CD99 expression in DIPG cells with radiation and that radiation-sensitization of DIPG cells to 10D1 CD99 antibody induces greater cell death.

FIG. 14 shows the anti-tumor efficacy of the 10D1-CD99 antibody in clearing Ewing Sarcoma (ES). Ewing sarcoma (A4573) tumor cells were implanted in the mouse femur and treated with 8 mg/kg of the antibody. Representative IVIS images are shown.

FIG. 15 shows low dose fractionated radiation-induced CD99 on Ewing Sarcoma (bone cancer in children) (ES) tumor cells.

FIG. 16 shows antagonizing CD99-sensitized DIPG tumors response to radiation treatment (RT), leading to prolonged survival in mice.

FIG. 17 presents an embodiment of the disclosed antibody heavy (top) and light (bottom) amino acid sequences: signal peptides are printed in black, CDRs in red, framework in the blue, and constant region in green.

FIG. 18 (Panel A) shows that treatment with CD99 (10D1) antibody did not affect normal human fibroblast (N2) cell proliferation and did not induce cell death (Panel B) Cell surface expression of CD99 in these cells as measured by flow cytometery.

FIG. 19 (Pane A) shows that treatment CD99 (10D1) antibody did not induce cell death in normal induced pluripotent stem cells (iPSC) derived from human skin. (Panel B) Flow cytometry plot showing the expression of CD99 in iPSC cells.

FIG. 20 shows the kinetics of peptide antigen interacting with 10D1-1 human IgG4/light chain1 using an SA biosensor. Association and dissociation of peptide antigen with 10D1-1 human IgG4/light chain 1 monitored by OctetRED 384.

### DETAILED DESCRIPTION

Whole-exome/genome sequencing studies on DIPG tumor samples reveal characteristic mutations in the H3.3, H3F3A, or HIST1H3B histone genes as well as several other epigenetically associated genes such as ATRX. Histone mutations, specifically mutations at position 27 result in a Lys to Met (K27M; or ‘H3K27M’) substitution and are found in 74-85% of all DIPG tumors. Conversely, another pediatric tumor, supratentorial, high-grade gliomas (HGAs), rarely possess these mutations (more frequently, these tumors show Gly34Arg/G34R or Gly 34Val/G34V substitutions).

The H3K27M mutation resulted in a global loss in H3K27me3 repressive mark leading to a dysregulation in the epigenome. However, overexpression of the H3K27M transgene by itself does not induce DIPG tumors when expressed in murine brain stem cells suggesting that additional factors are cooperating with the oncogenic H3K27M mutation to accelerate neoplastic transformation.

Applicants' focus has been on identifying and targeting the factors and mechanisms that may cooperate with H3K27M mutations that can lead to new therapies for DIPG, which, as noted above, is an otherwise refractory disease.

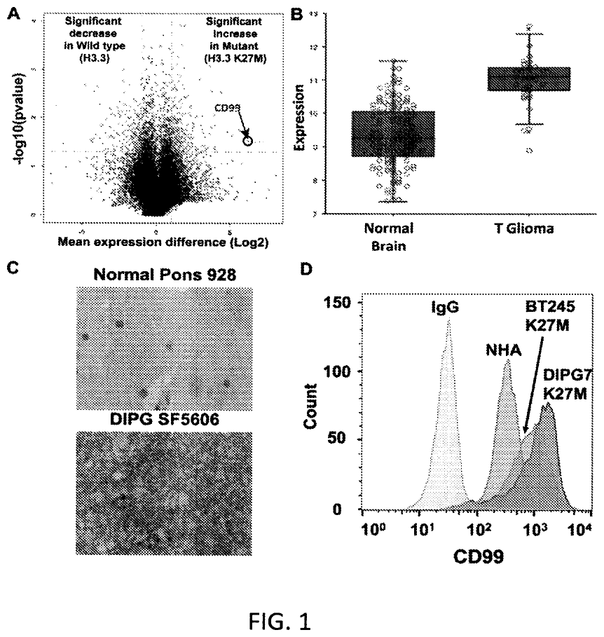

Applicants performed RNA sequencing analysis of paired tumor samples (one histone mutant and one wild-type), which revealed a significant upregulation of a specific gene. The gene, MIC2/CD99, was upregulated in the samples expressing H3K27M mutations compared to non-mutant wild-type samples (See FIG. 1 Panel A). High expression of CD99 was seen in DIPG patient tumors at the transcriptomic and at protein levels (FIG. 1 Panels B and C).

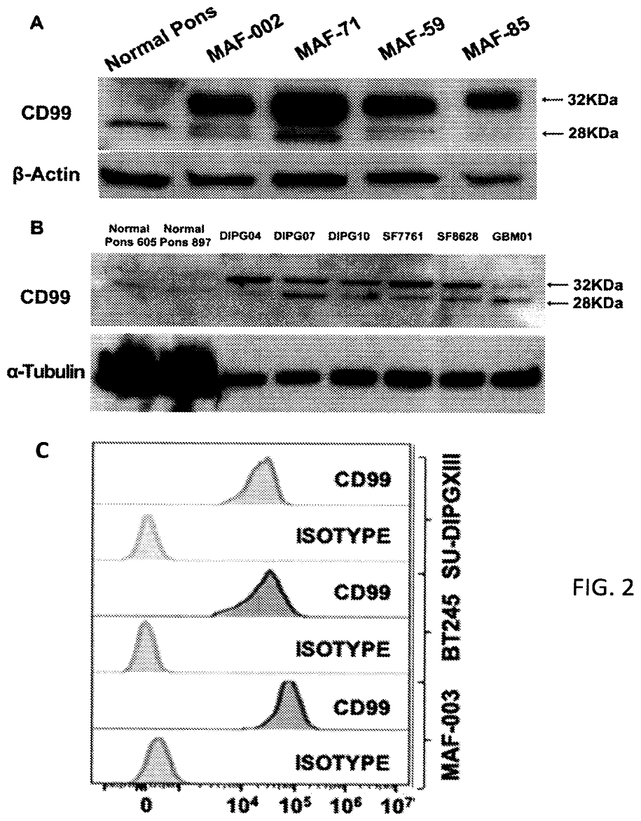

DIPG tumor cells express high levels of CD99 compared to normal human astrocyte (NHA) cells as measured by flow cytometry (FIG. 1 Panel D). Single-cell RNA-sequencing of the H3K27M DIPG patient tumors showed significantly elevated levels of CD99 in the neoplastic population of these cells. Applicants have identified differential expression of two dominant expressing CD99 isoforms—a long form and a short form. Immunoblotting analysis of multiple DIPG patient tumor samples revealed that CD99 is highly expressed in DIPG tumor cells compared to normal pontine cells (FIG. 2 Panel A) and that the long form of CD99 (the active form) is predominant in DIPG tumors both in the patient samples and in tumor cell lines while the short form (inactive form) was preferentially expressed in the normal cells. The expression of the short form was much less compared to its long-form counterpart in the tumor cells (FIG. 2).

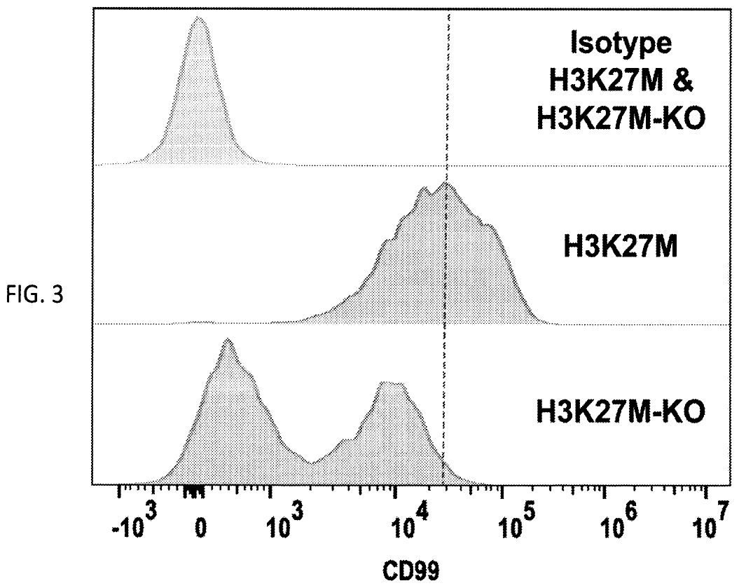

The overall survival analysis in DIPG patient cohorts revealed that higher proportions of CD99 are associated with the worst prognosis. Deletion of the H3K27M mutation by CRISPR/Cas9 technology resulted in a complete loss of CD99 (FIG. 3), further substantiating the importance of CD99 in H3K27M DIPG tumors.

To understand the biology of the H3K27M mutation, isogenic GBM cells expressing the H3K27M mutation were created. RNA sequencing was then performed on the paired samples. Differential expression analysis showed a several-fold increase in CD99 in cells overexpressing the mutant H3K27M compared to the non-mutant expressing WT cells. We then examined the expression of CD99 in the H3K27M mutant expressing DIPG patient tumors and cell lines.

DIPG patient tumors showed elevated expression of CD99 at the mRNA level (available at hgserver1.amc.nl/cgi-bin/r2/main.cgi) and at the protein level compared to that of normal pons by IHC. Similarly, elevated expression of CD99 was detected in the patient-derived DIPG cell lines compared to normal human astrocytes as measured by Flow cytometry. Genetic knockdown of CD99, decreased tumor initiation in mice suggesting CD99 as a therapeutic target in DIPG. Applicants hypothesized that blocking CD99 using an anti-CD99 antibody would be an effective anti-tumor therapy in DIPG.

Therapeutic anti-CD99 antibodies, to be effective, would need to be capable of crossing the Blood-Brain Barrier (BBB). or be delivered other than systemically. Commercially available anti-CD99 antibodies show poor brain penetrant capacity and are not therapeutic. For at least these reasons. Applicants created novel anti-CD99 antibodies against a 15 amino acid sequence of H3K (SEQ ID NO:1). Applicants have tested the presently disclosed antibody for efficacy in vitro and in vivo. The specificity of the disclosed antibody has also been analyzed for the ability to detect cell surface CD99 protein by immunohistochemistry (IHC). Detection of CD99 can be used as a prognostic marker for DIPG.

### The H3K27M, Onco-Histone Mutation Cooperates with CD99 in Driving DIPG Tumorigenesis

RNA sequencing analysis of paired tumor samples (i.e. H3K27M mutant and H3-wildtype) revealed significant upregulation of the gene MIC2/CD99 in the H3K27M-expressing samples compared to non-mutant wildtype samples (FIG. 1A). High expression of CD99 was also detected in DIPG patient tumors at both the transcriptomic and at protein levels (FIG. 1 Panels B and C). Flow cytometry confirmed that DIPG cells express high levels of CD99 compared to NHA cells (FIG. 1 Panel D). Single-cell RNA-sequencing of H3K27M DIPG patient tumors showed a significantly elevated level of CD99 in the neoplastic population of these cells.

Applicants identified, for the first time, differences in the expression of two pre-dominantly expressed CD99 isoforms—a long/active form and a short/inactive form. Immunoblotting analysis of multiple DIPG patient tumors revealed that CD99 is highly expressed on DIPG tumor cells compared to normal pontine cells (FIG. 2 Panel A) and that the long form of CD99 (i.e. the active form) is the predominant form in DIPG tumors both in the patient samples and in tumor cell lines: in contrast, the short form (inactive form) was the predominant form expressed in normal cells. The expression of the short form was much less than its long-form counterpart in the tumor cells (FIG. 2).

The overall survival analysis in DIPG patient cohorts revealed that higher proportions of CD99 are associated with the worst prognosis. Deletion of the H3K27M mutation by CRISPR/Cas9 technology resulted in a complete loss of CD99 (FIG. 3), further substantiating the importance of CD99 in H3K27M DIPG tumors.

### Genetic Inhibition of CD99 Impedes DIPG Tumor Initiation in Animal Models

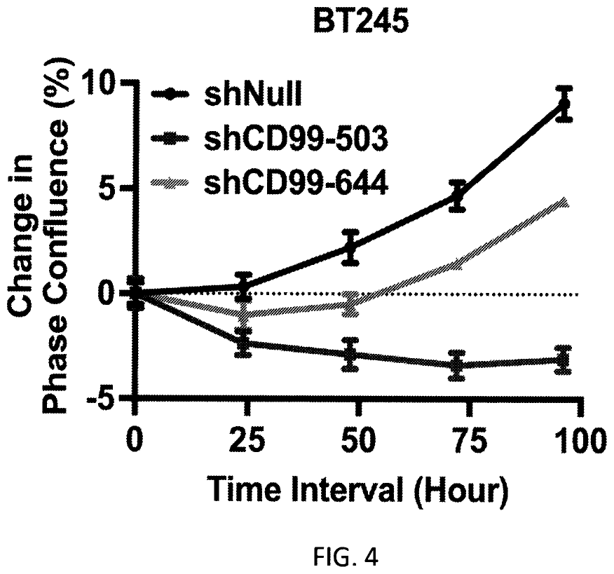

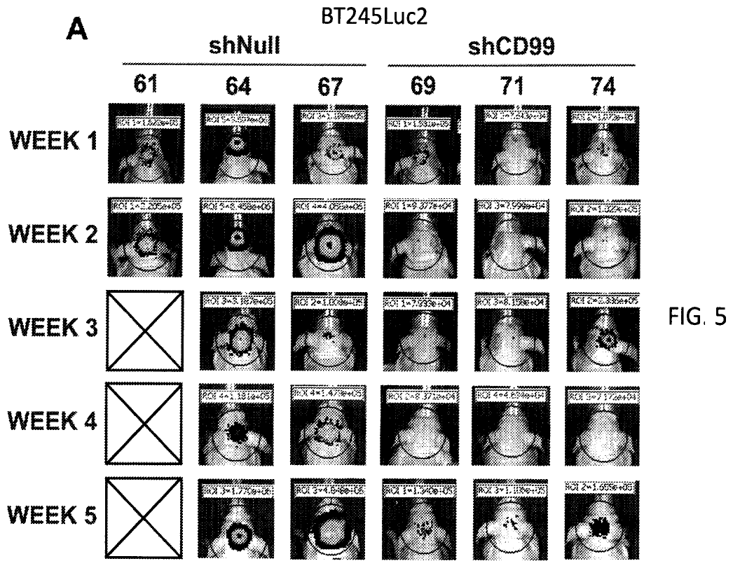

Next, Applicants investigated the ability of CD99 to play an oncogenic role. In these experiments, CD99 expression was depleted using ShRNAs targeting CD99. In these experiments, in-vitro growth analysis showed a significant decrease in tumor growth with CD99 knockdown (FIG. 4).

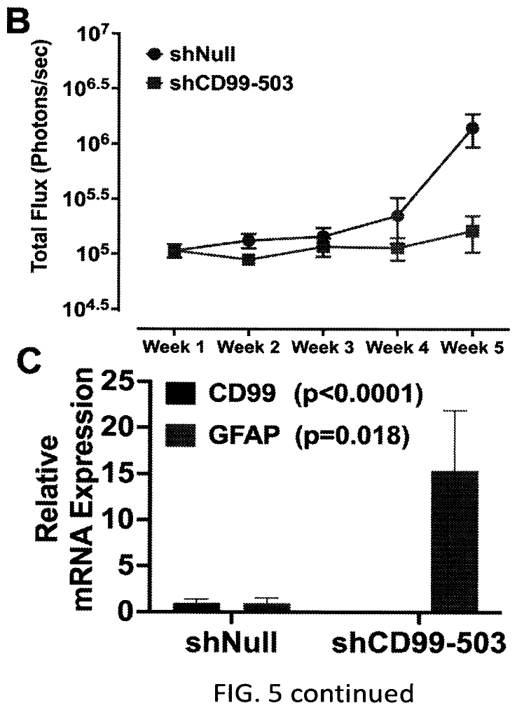

The tumorigenic capacity of the DIPG CD99 knock-down cells was tested in vivo. For these experiments, CD99-sufficient and CD99-depleted luciferase expressing DIPG tumor cells in the mouse pons and monitored tumor growth in mice by measuring the luciferase bioluminescence (IVIS) every week. We found that while the DIPG tumor cells expressing CD99 (shnull, control) established tumor in the pons within ten days after the tumor implantation, the CD99 depleted tumor cells showed delayed latency in tumor establishment, suggesting that CD99 is vital in the initiation or onset of tumorigenesis (FIGS. 5A and B). Additionally, these delayed tumor establishment results were associated with an increase in differentiation, thereby blocking rapid growth of the tumor (FIG. 5C).

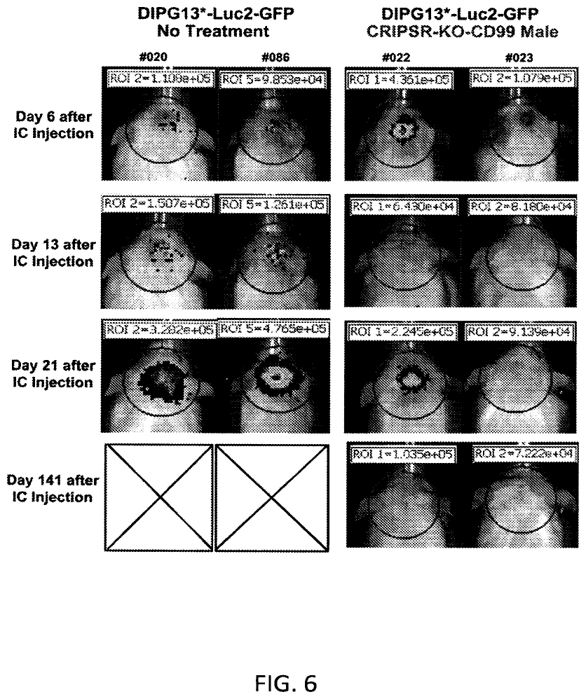

In parallel with tumorgenicity studies, Applicants generated CRIPSR-mediated complete deletion of the CD99 gene. These experiments targeted the CD99 gene in DIPG cells. When implanted, these CD99 deletion cells failed to establish a tumor in the mouse pons. These results further support a central role for CD99 in DIPG-associated tumor establishment and/or growth (FIG. 6).

### Rationale Design and Synthesis of Novel Chimeric CD99 Antibody

The ability of an antibody to block CD99 and phenocopy the genetic knockdown of CD99 discussed above was tested. A commercially available anti-CD99 antibody (clone 0062) was tested first. Treating H3K27M-mutant DIPG cells with this anti-CD99 antibody reduced DIPG cell growth as measured by real-time cell growth assay. This suggests that CD99 plays an important role in DIPG tumor cell proliferation and that blocking CD99 using antibody can impede cell growth. Commercially available research-grade anti-CD99 antibody were then tested for its in vivo efficacy against DIPG.

Further investigation of the commercially available antibody, clone 0662, indicated it is an IgG3 antibody. Studies indicated that among the four subtypes of IgGs, namely IgG1, 2, 3, and 4, IgG3 immunoglobulins have poor characteristics, high molecular weight and polymeric nature, for use in vivo on brain tumor model studies. These characteristics may hinder IgG3's ability to cross the blood-brain-barrier (BBB). Similarly, the 0662 antibody also demonstrated a moderate capacity to cross the BBB.

For at least these reasons, Applicants synthesized a clinically relevant, novel CD99-targeting antibody.

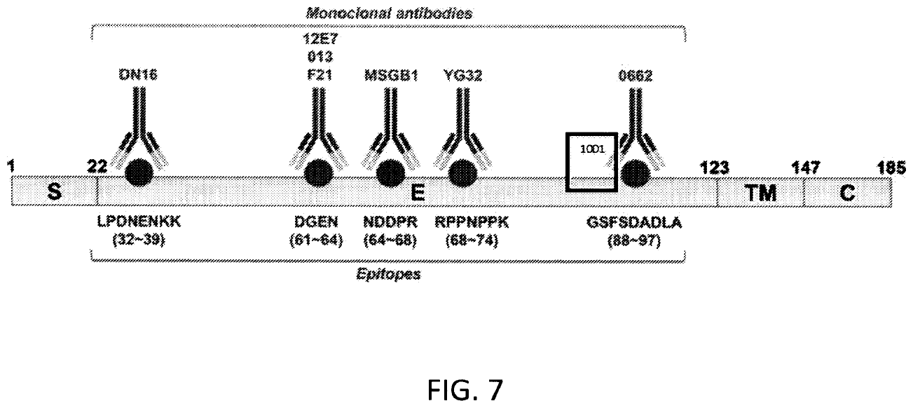

FIG. 7 shows the commercially available murine monoclonal CD99 antibodies and their corresponding epitope binding regions in the CD99 protein. Most commercial anti-CD99 antibodies target epitopes within the N-terminal region of the extracellular domain of CD99. One exception is the 0662 clone, which targets an epitope at the C-terminus of this same domain without wishing to be restricted by theory, this may be to increase binding and stability.

The applicant's presently disclosed CD99 antibody is targeted to a novel CD99 epitope. This epitope is shown in red in FIG. 7. Specifically, the presently disclosed 15 amino acid epitope sequence, SEQ ID NO: 1, is at the C-terminus of the CD99 protein. The presently disclosed monoclonal anti-CD99 antibody clone, referred to as 10D1, possesses high binding affinity and specificity for human CD99: comprising CDRs of SEQ ID NO: 6,7, 8, 10, 11, and 12. In various embodiments, the variable domains comprise polypeptides of SEQ ID NO: 2 and SEQ ID NO: 3, which may be coded for by SEQ ID NO: 5 and SEQ ID NO: 9. In some embodiments, the Light Chain is a polypeptide of SEQ ID SEQ ID NO: 15, and may be coded for by a polynucleotide of SEQ ID NO: 16. In some embodiments the Heavy Chain is a polypeptide of SEQ ID NO: 13, and may be coded for by a polynucleotide of SEQ ID NO: 14. In many embodiments, the Fc Domain is human. In many embodiments, the Fc Domain is an IgG4 Fc domain. In some embodiments, the Fc Domain is a polypeptide of SEQ ID NO: 4.

A recombinant protein also was made using the above sequences but comprising an Fc region of human IgG4. The presently claimed CDRs and/or variable domains may possess reduced toxicity and/or increased ability to cross the BBB. Our CD99 antibody will be referred from here on as 10D1-CD99 antibody.

### Specificity and Cytotoxic Effect of 10D1-Anti-CD99 Antibody

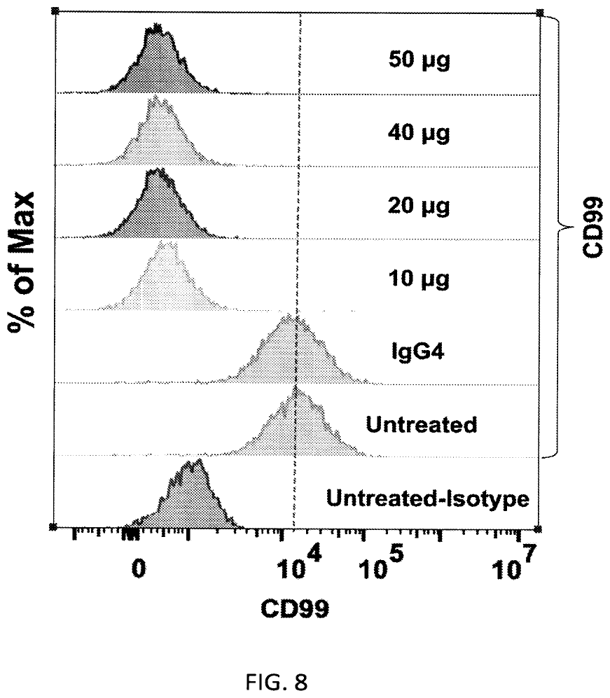

The engagement/binding of the presently disclosed 10D1-CD99 antibody to the human CD99 epitope was tested. In these experiments, DIPG tumor cells were incubated with the 10D1-CD99 antibody for 30 minutes and cells were then analyzed by flow cytometry using the commercial 0662 CD99 antibody. FIG. 8 shows that even the use of low amount of 10D1-CD99 completely blocked CD99 expression in these cells suggesting the high affinity of the 10D1-CD99 antibody binding to human CD99.

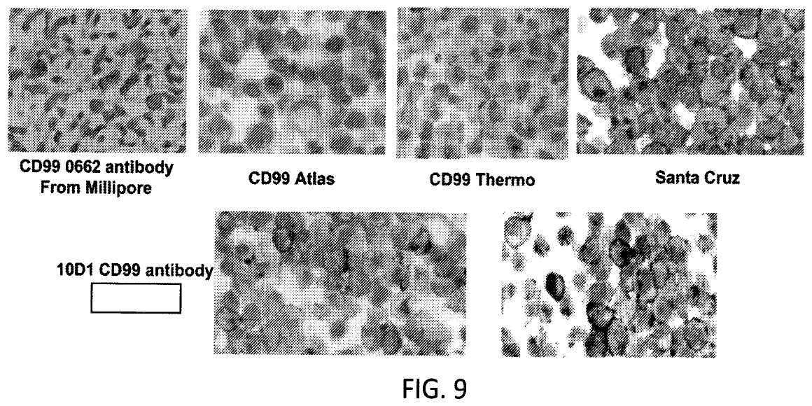

The 10D1-CD99 antibody was tested for its ability to detect CD99 on patient samples by performing immunohistochemical staining (IHC), and comparing the results to other commercially available antibodies. The 10DI-CD99 antibody stained specifically the CD99 protein on the cell surface, while most other commercial antibodies showed weaker staining, and the most commonly used antibody (Santa Cruz) showed non-specific staining of CD99 in the nucleus (FIG. 9). This suggests that the 10D1-CD99 antibody is superior to commercially available antibodies in having specificity to the cell surface protein, CD99.

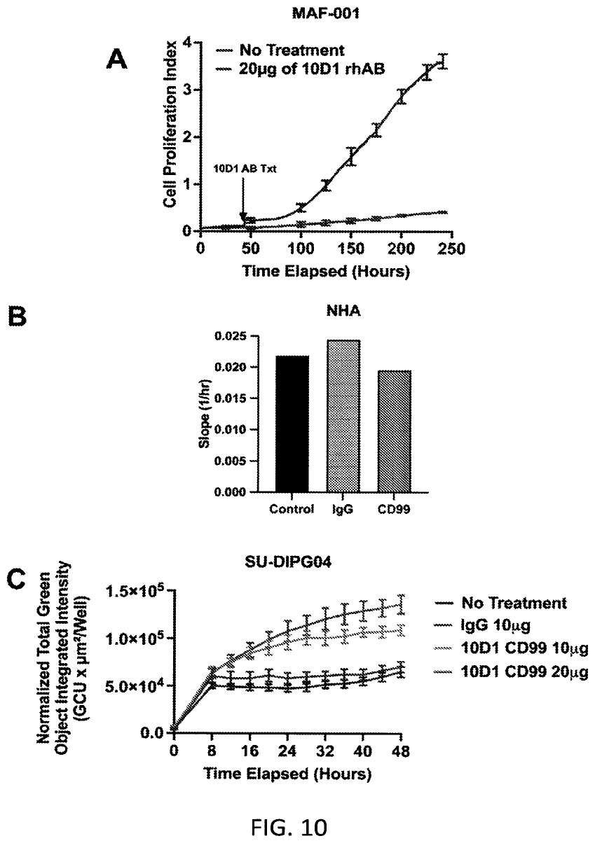

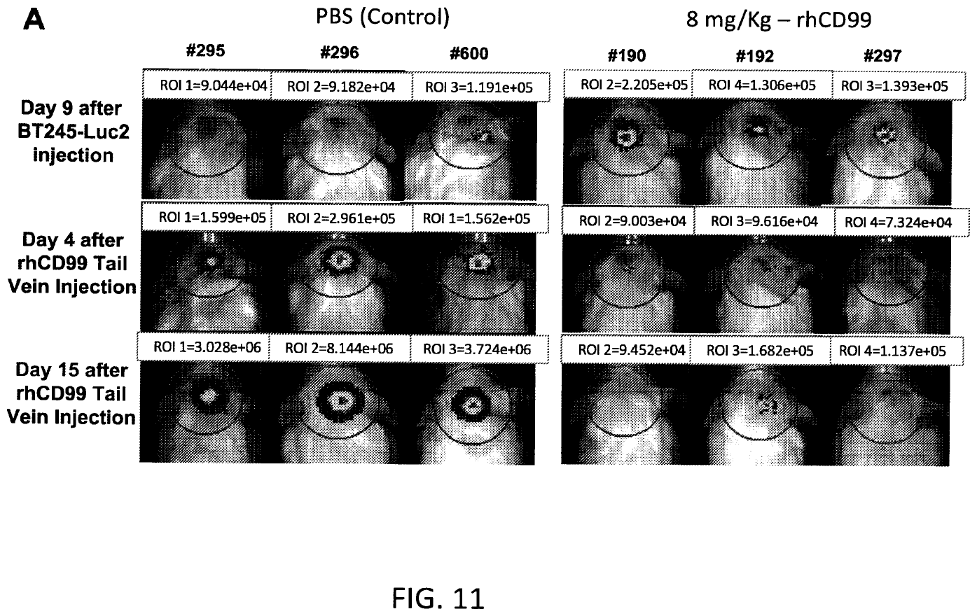

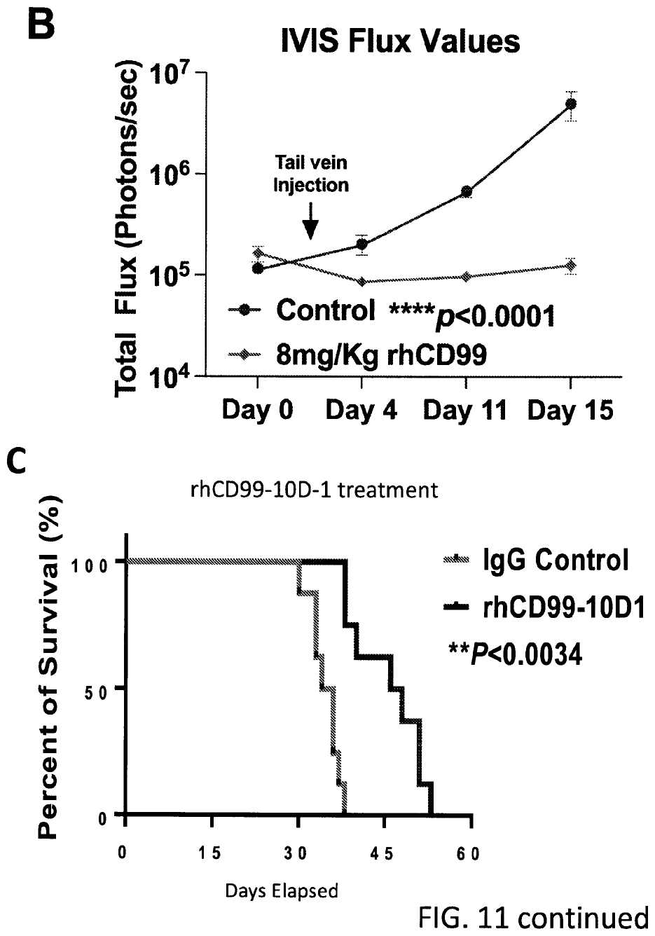

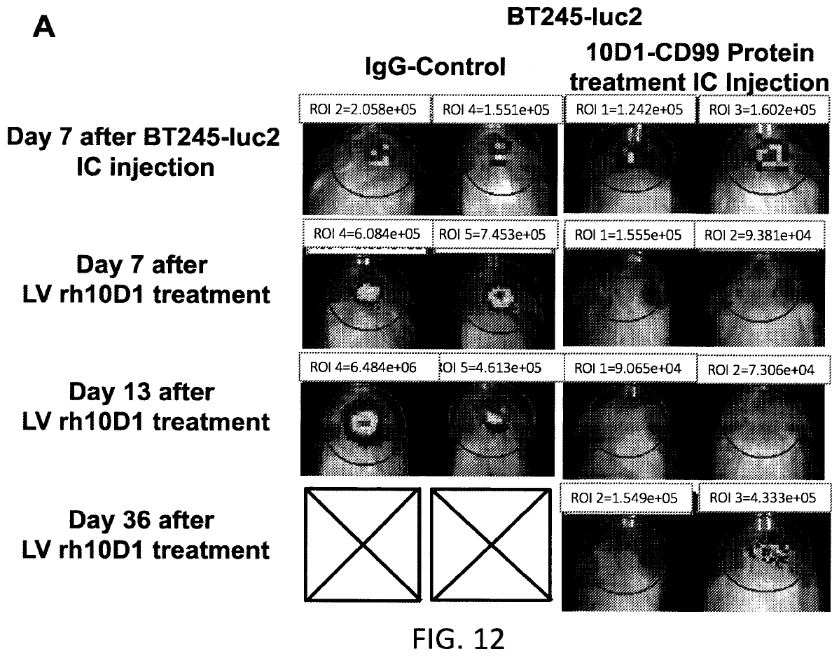

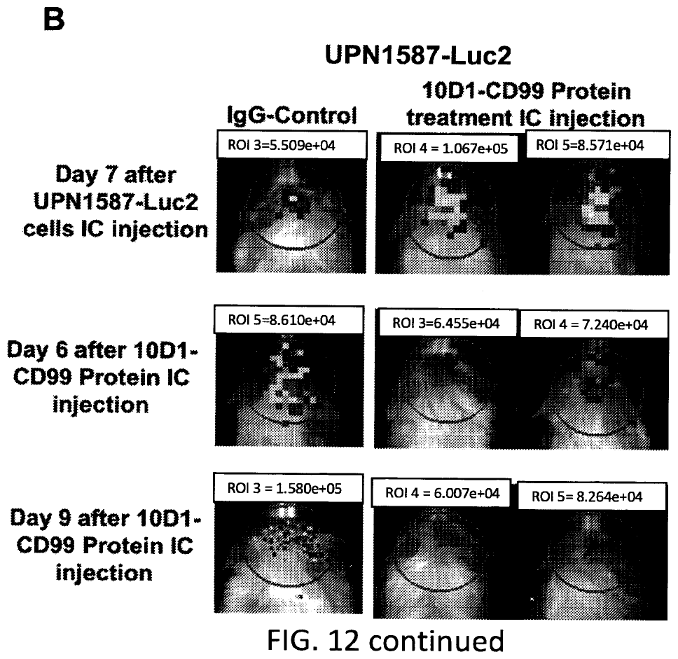

The 10D1-CD99 antibody was tested for cytotoxicity against DIPG tumor cells. Treatment of DIPG cells with 10D1-CD99 antibody resulted in a significant decrease in cell growth (FIG. 10A, B) and a concomitant increase in cell death (FIG. 10C). This strongly suggests that the 10D1-CD99 antibody is functional as expected and are highly specific in targeting human CD99.

The disclosed compositions and methods may be useful in treating a variety of diseases and conditions. for example, cancer and cells associated therewith (i.e. cancer cells and tumor cells). Cancer, cancer cells, tumor, and tumor cells as used herein, may refer to various diseases and conditions, such as pediatric and adult cancers, as well as solid and liquid tumors associated therewith. In one example, cancer may be brain cancer. In many embodiments, the disease or condition may include, without limitation, acute myeloid leukemia (AML), neuroblastoma, ependymoma, Ewing Sarcoma, diffuse intrinsic pontine glioma (DIPG), and the like.

### Therapeutic Window in Targeting Only DIPG Tumor Cells with 10D1-CD99 Antibody

The application of immunotherapy to solid tumors may be hindered by the lack of “tumor-only” antigens. Since other normal cells also express CD99 (although at low levels compared to CD99 expression in tumor cells), the effect of the 10D1-CD99 antibody on normal human astrocytes (NHA) was tested. As shown previously (FIG. 1D). expression of CD99 is high in tumor cells but comparatively low in NHA cells. NHA cells, when treated with 10D1-CD99, antibody showed little to no change in viability. suggesting that blocking CD99 specifically inhibits tumor cell growth while having little effect on normal cells. These results indicate the presence of a therapeutic window in targeting high CD99 expressing DIPG cells (FIG. 10 Panel D).

### Antitumor Efficacy of rh10D1 Anti-CD99 Antibody Against DIPG in Xenograft Models

Applicants developed the presently disclosed novel antibody to promote efficient and specific crossing of the antibody through the BBB. In many embodiments, the target epitope sequence was selected to help minimize coagulation. In many embodiments. the target epitope sequence was selected to help increase binding affinity. In many embodiments, the disclosed anti-CD99 antibody possess an IgG4 domain to minimize size and aid in crossing the blood-brain barrier.

The disclosed anti-CD99 antibody may be administered to a patient by various methods. In some embodiments, the disclosed anti-CD99 antibody may be delivered directly into the intracranial regions of a patient. These methods may aid in lowering the amounts and/or concentrations of the disclosed antibody necessary. For example, in some embodiments, wherein the antibody is delivered intracranially, approximately 1/10^{th} the amount of a dose for I.V. administration may be required dose—in the case of a mouse, for example, 20 ug vs. 200 ug/mouse/day may be required. In other embodiments, a single dose of the antibody may be necessary, where 8 doses may be necessary when administration is via I.V. In these embodiments, intracranial delivery may reduce the amount and the cost required to treat a subject. In many embodiments, intracranial administration may aid in reducing toxicity, if any, to normal cells. In many embodiments, a dose of the disclosed antibody may be significantly diluted in the blood stream when the subject is treated with a single, low dose of the disclosed anti-CD99 antibody. Alternative delivery may include convection enhanced delivery or Convection Enhanced Delivery (CED). CED was tested in the mouse xenograft models disclosed below. CED involves delivery of a therapeutic, in one embodiment the disclosed anti-CD99 antibodies (wherein dosing may be similar to the above intracranial delivery) directly to the tumor location site, here the pons.

### Adjuvant Radiation Therapy

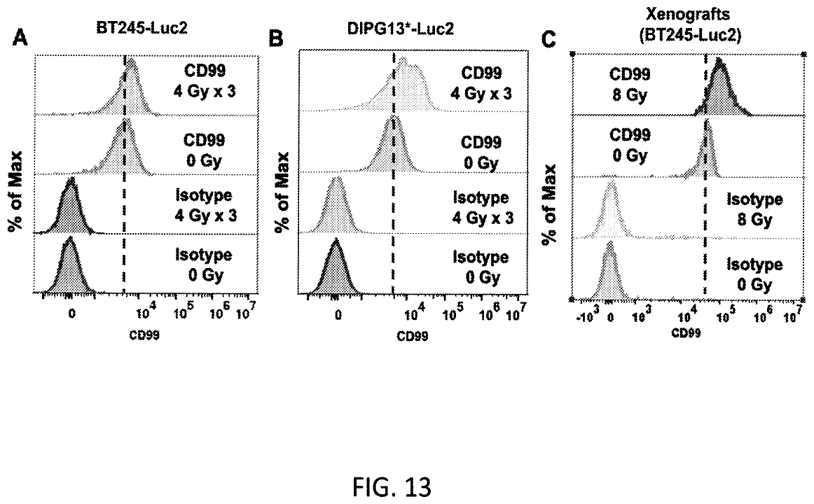

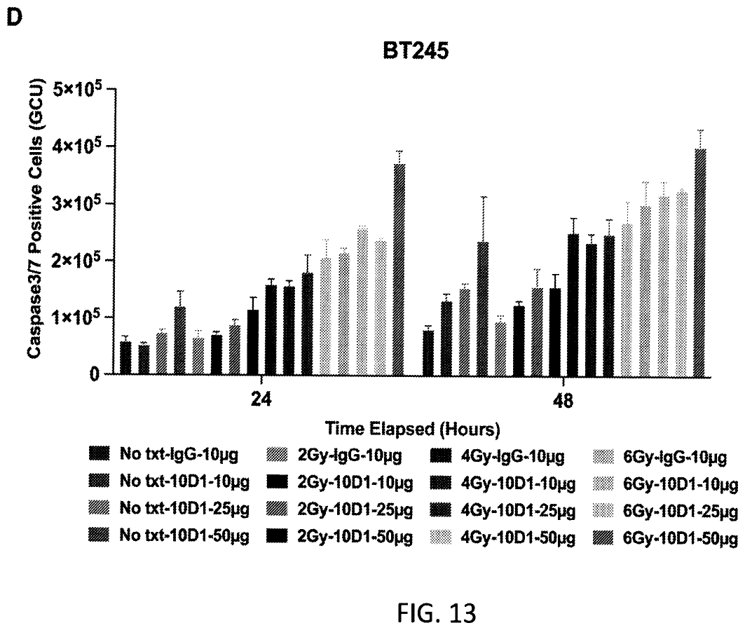

Radiation is currently the only standard of care for DIPG patients. In many embodiments, treatment with the disclosed anti-CD99 antibody may synergistically aid radiation treatment. Specifically, at FIG. 13. Applicants show that fractionated radiation treatment of DIPG tumors increases expression of CD99 on tumor cell surface (FIG. 13A), making these cells a better target for the disclosed anti-CD99 antibody. In some embodiments, radiation treatment combined with anti-CD99 antibody therapy may help to broaden the therapeutic window. In these embodiments, combining the disclosed anti-CD99 antibody treatments with radiation may aid in specifically targeting the high CD99 expressing tumor cells while protecting the normal cells.

Applicants disclose, herein. exposing DIPG cells to fractionated radiation and subsequently treating the irradiated cells with the disclosed 10D1-CD99 antibody. In these experiments, tumor cell death was measured by analyzing changes in the active caspase 3/7 using incucyte live cell imaging. Applicants found that fractionated radiation increased CD99 induced cytotoxicity of DIPG tumor cells (FIG. 13B), demonstrating that fractionated low-dose radiation synergizes with the disclosed 10D1-CD99 antibody to increase DIPG tumor cell death.

### Use of the Disclosed 10D1-CD99 Antibody to Treat Pediatric Cancers

Ewing sarcoma is a rare devastating tumor of the bone in children. Approximately, 25 to 30% of ES patients presents with evidence of metastases at diagnosis. Children and young adults with relapsed and/or metastatic Ewing Sarcoma(ES) have a very poor prognosis despite intensive treatment with traditional chemotherapy, radiation, and surgery. There have been no therapeutic advances for these patients for the past four decades, highlighting the critical need for novel approaches to treat metastatic and recurrent ES.

CD99 is highly expressed on the surface of ES tumors, and blocking CD99 decreased ES tumor cell growth (Scotlandi K, et al., Cancer Res 2000, 60(18):5134-5142). Applicants found that treating Ewing Sarcoma cells with our therapeutic 10D1 antibody showed decreased tumor cell proliferation significantly. Therefore, we hypothesize that our 10D1 chimeric antibody can be used to treat ES tumors in children.

### Anti-Tumor Efficacy of Novel rh10D1-CD99 Anti-CD99 Against Pediatric Ewing Sarcoma (ES) Model

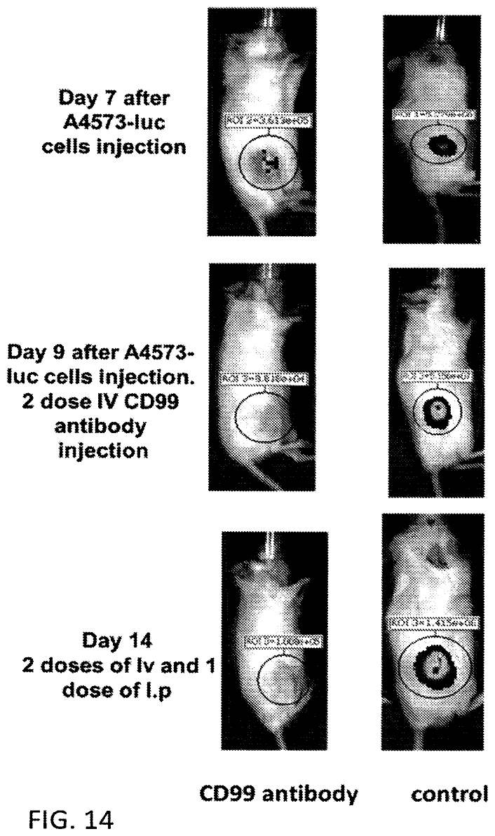

First to test the applicability of 10D1-CD99 antibody to pediatric ES tumor treatment, we performed an initial in vivo experiment to identify the efficacy of our rh10D1 anti-CD99 antibody in clearing ES tumor burden. Treatment of mice with established primary ES tumor with rh10D1 antibody with 3 doses of 8 mg/Kg by I.V. on alternate days and 3 doses of I. V. thereafter for 3 alternate days cleared tumor burden and prolonged survival to greater than 80 days (FIG. 14). This suggests that the recombinant 10D1-CD99 antibody is active against ES and are more effective in treating solid tumors like DIPG and ES.

### Fractionated Radiation Induced CD99 on DIPG Tumor Cells and Therefore can Sensitize Tumor Cells to 10D1-CD99 Antibody Treatment

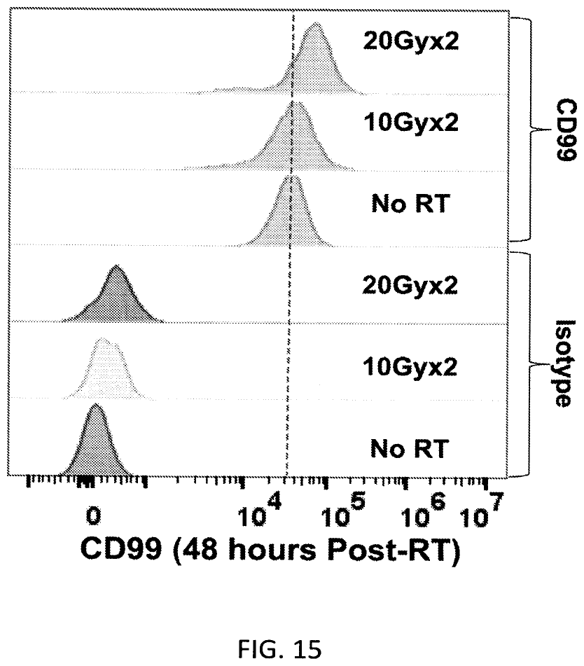

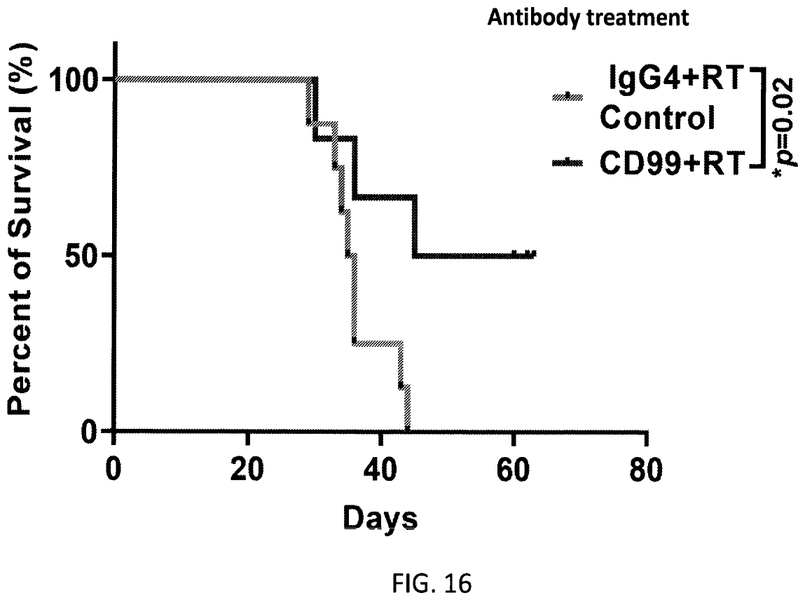

FIG. 16 shows survival curves for DIPG tumor bearing mice treated with the disclosed 10D1-CD99 antibody followed by radiation as a combination treatment. Antagonizing CD99 sensitized DIPG tumor cells to radiation thus leading to prolonged xenograft survival. Similar to DIPG tumor cells, Ewing sarcoma tumor when exposed to fractionated radiation showed increased levels of CD99 (FIG. 15). Therefore, similar sensitization of Ewing Sarcoma cells to radiation treatment when combined with 10D1-CD99 antibody.

For the studies shown in FIG. 16, Human DIPG (or DMG) cells tagged with luciferase-GFP (BT245-Luc2-GFP) were implanted in the pons of 6- to 8-week-old male and female NSG mice. Briefly, a suspension of ˜1×105 cells in 2 μl serum-free media were stereo tactically injected at a rate of 500 nL/min into the brain at a site 0.8 mm lateral to midline, 0.5 mm posterior to lambda, and 5.00 mm ventral to the surface of the skull. Tumor formation was monitored by bioluminescent imaging (BLI) once per week using IVIS Xenogen 2500 imaging machine. After conformation of tumor establishment in the pons, with BLI corresponding to ˜105 to 106 photons using IVIS, animals were randomized into 2 treatment groups as follows: (1) IgG4 +RT (control), n=7; (2) 10D1-CD99 antibody +RT n=6, administered intra venously. Antibodies were dosed at 8 mg/kg body weight/day for 5 days followed by fractionated focal radiation treatment at 2 Gy/day for 3 consecutive days. Details of radiation treatment and the radiation source used are under in vivo radiation section. Tumor growth and response to therapy were determined biweekly by BLI imaging. The tumor take rate was 100%. Body weight was measured once a week and mice were monitored daily and those reaching end-point were euthanized according to IACUC protocols by CO2 asphyxiation. when they show signs of either neurological deficit. failure to ambulate, body score less than 2, or weight loss greater than 20%. Animal survival curves were analyzed using Kaplan-Meier method and statistical significance (p<0.05) was computed using Gehan-Breslow-Wilcoxon tests, with groups compared by respective median survival or number of days taken to reach 50% morbidity.

### In Vivo Radiation Method

Animals received a fractionated doses of 2 Gy per day for 3 consecutive days. Under isoflurane anesthesia, each mouse is positioned in the prone orientation and aligned to the isocenter in two orthogonal planes by fluoroscopy. Each side of the mouse brain received half of the dose is delivered in opposing. lateral beams. Dosimetric calculation was done using a Monte-Carlo simulation in SmART-ATP (SmART Scientific Solutions B. V., Maastricht. the Netherlands) for the 4^{th} ventricle+Mid Brain+Pons receiving the prescribed dose. Treatment was administered using a XRAD SmART irradiator (Precision X-Ray. Madison CT) using a 225 kV photon beam with 0.3 mm Cu filtration through a circular 10 mm diameter collimator.

The radiosensitization experiment was performed to investigate the ability of high expression of CD99 on DIPG tumor cells to protect the cells from radiation therapy causing tumor to relapse. In these studies, CD99 was blocked using anti-CD99 antibody first and then cells were subjected to radiation therapy. The results demonstrate that blocking CD99 followed by RT can significantly increase DIPG xenograft survival.

Next whether inhibition of CD99 after radiation treatment can induce greater sensitization as it abrogates any treatment resistance induced by CD99 was investigated. For this analysis. in vivo experiments were performed in which DIPG xenografts were first exposed to fractionated radiation (2 Gy/day for 3 consecutive days) followed by anti-CD99 antibody (10D1) treatment dosed at 8 mg/kg/day for 5 days. Animal survival outcomes were then determined.

Multiple methods of combining radiation therapy (RT) with the disclosed anti-CD99 antibody are envisioned. In some embodiments, radiation and the disclosed anti-CD99 antibodies may be delivered concurrently, for example where radiation and anti-CD99 (for one example 10D1) antibody may be administered concurrently under the following conditions: 1) RT and 10D1 antibody administered on the same day for 3 days: 2) 10D1 antibody pre-treatment followed by administration of RT and 10D1 antibody the same day for 3 days: 3) Pre-treatment with 10D1 antibody followed by RT and 10D1 antibody and continue antibody post-treatment for 3 days. Antibodies may be administered intravenously, intercranially, or via CED.

Intracranial delivery of the disclosed anti-CD99 antibodies (e.g. 10D1) are very effective even at low dose in clearing the tumor. In many embodiments, the disclosed antibody delivered directly to the brain region at 1 mg/ml, for example at a single dose in combination with radiation (either before or after RT) may have greater anti-tumor impact compared to the other treatment methods.

The disclosed anti-CD99 antibodies, for example the 10D1 CD99 antibody, has demonstrated anti-tumor effects against DIPG in vitro and also in vivo. These results suggest that the presently disclosed antibodies cross the BBB. This ability to cross the BBB may help to overcome one obstacle in treating brain tumors.

Combining 10D1 CD99 antibody with radiation treatment enhances the anti-tumor efficacy. In various embodiments of the disclosed combination therapy radiation therapy can be applied before, after, concurrently, or a combination thereof, with the antibody therapy.

Direct delivery of 10D1 CD99 antibody was shown to result in greater anti-tumor effectiveness against DIPG tumors in vivo, even at low antibody doses. This result suggests loco-regional delivery can be effective in treating brain tumors with the disclosed anti-CD99 antibody therapies, for example, 10D1 CD99 antibodies.

The disclosed therapies, including therapy with the disclosed anti-CD99 antibody, for example, 10D1, can be used to treat a variety of indications and tumor types, especially in children. Potential indications and tumor types may include, without limitation, DIPG, Ewing Sarcoma, ependymoma and AML.

### CD99 as a Biomarker/Prognostic Marker for Solid Tumors like DIPG and ES

Radiotherapy is a standard treatment for DIPG and about 80% of the patients show a temporary tumor reduction and clinical improvement. while the rest 20% have no radiation benefit. We further discovered that DIPG tumor cells upregulate CD99 in response to radiation therapy as a protective mechanism to avoid radiation-induced cell death. Therefore, the expression of CD99 on the tumor cells can be used as a predictive marker of radiation response.

We have stained DIPG tumor tissues from a cohort of H3K27M and wildtype samples. CD99 is expressed in more than 90% of tumor cells, and tumor cells harboring H3K27M mutant express CD99 at higher levels than the wild-type cells. IHC may be performed with various embodiments of the disclosed anti-CD99 antibody, for example. the mouse 10D1-CD99 monoclonal antibody specific to CD99. H3K27M tumors may be examined for expression levels and uniformity of CD99 on the cells. Comparing tumor tissues that have been exposed to radiation and radiation-naïve paired tumors. Applicants may be able to determine a protective mechanism of tumor cells by CD99 upregulation.

Additionally, previous studies have shown the detection of CD99 in circulating tumor (Ct) cells as a prognostic marker of risk and treatment efficacy in the blood samples of Ewing sarcoma patients [16]. Recently a breakthrough study prompted that this methodology can be applicable in DIPG patients. In this study. Applicants show that the presence of H3K27M mutation can be quantitatively detected in circulating tumor cells in blood from DIPG patients [17]. Taken together, Applicants propose detection of CD99, using an embodiment of the disclosed anti-CD99 antibody, and in some cases H3K27M, in Ct DNA in blood samples of DIPG patients to aid in developing prognosis and treatment predictive outcomes. In some embodiments, a similar methodology can be applied to identify CD99 along with EWS-FLI fusion, (a characteristic genetic alteration in ES) in circulating tumor cells in blood from ES patients.

### Treatment of Normal Cells with Anti-CD99 Antibodies

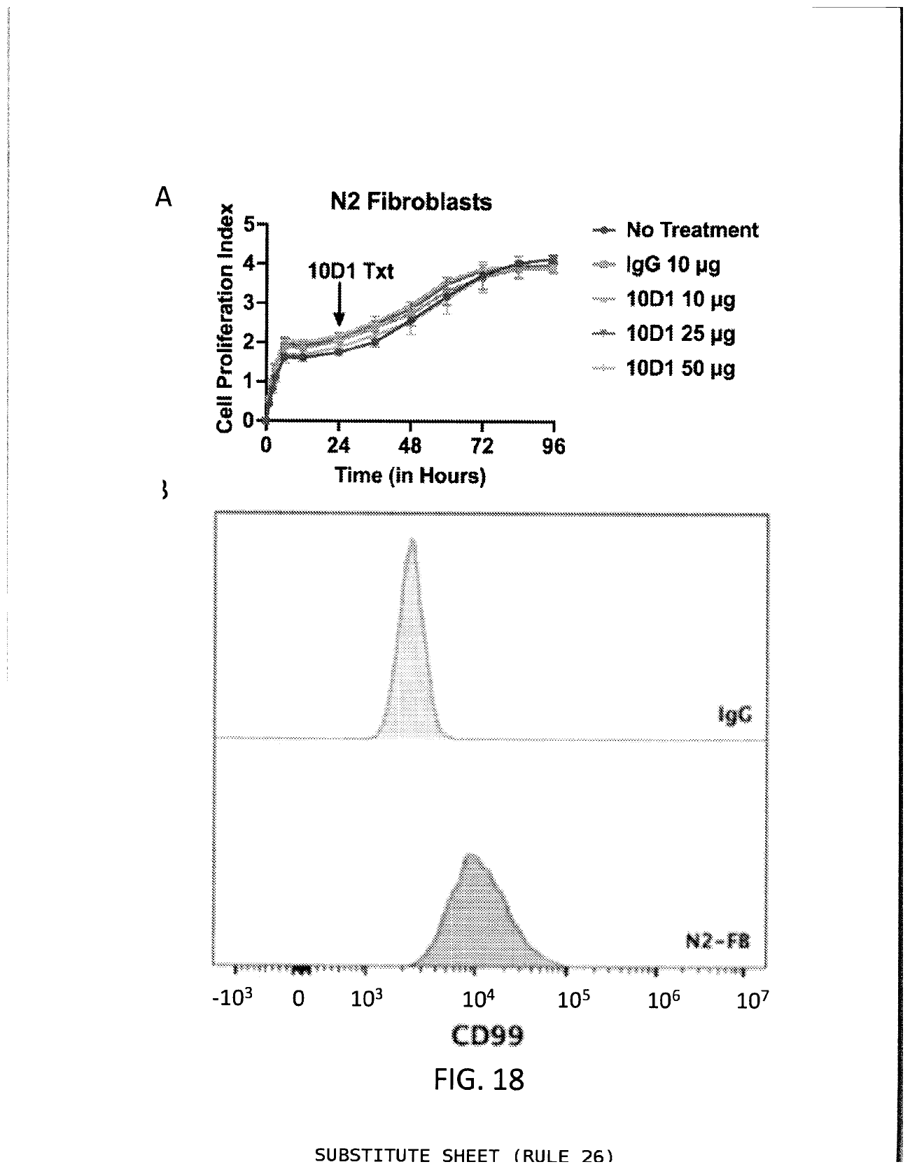

Normal human fibroblasts (N2) were exposed to the presently claimed anti-CD99 antibodies. FIG. 18 shows that that cell proliferation was not affected (Panel A), and cell death was not induced (Panel B). Panel A is results from a XCELLignece real time growth assay, where fibroblast (N2; specifically Normal Human Neonatal Dermal Fibroblasts, Lonza CC-2509) cells were seeded onto the 96 well-gold coated xcelligence plate on day 1 and 24 hours later treated with either IgG 4 control or 10D1 CD99 antibody, while cell proliferation was monitored over time. Panel B is results from flow cytometry plotted and showing the expression of CD99 (expressed, but at low levels) in N2 fibroblast.

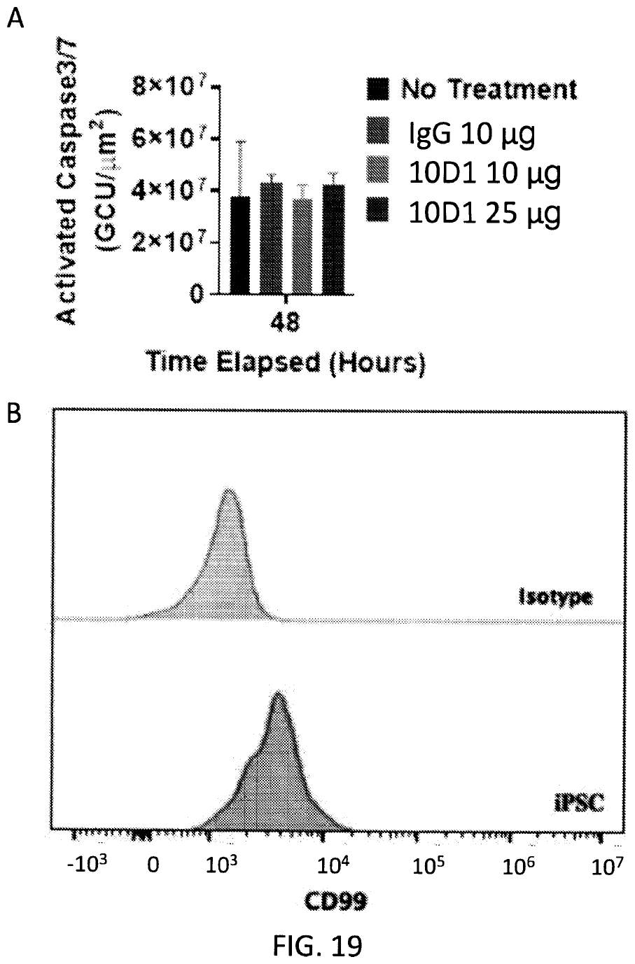

Normal induced pluripotent stem cells (iPSCs) derived from human skin were treated with anti-CD99 antibodies. FIG. 19 shows results from these studies demonstrating that the disclosed anti-CD99 antibodies did not induce cell death. Panel A presents results from an. apoptosis assay. Here. IPSC, derived from normal donor skin, was cultured and treated with either control IgG4 or with two different concentrations (10 μg and 25 μg) of the 10D1 anti-CD99 antibody. Cell death was monitored by incucyte, a live cell imaging system in the presence of green caspase3/7 reagent in the hypoxic incubator maintained at 2.5% O2. The changes in the caspase3/7 activity, as measured by changes in the green caspase, were measured after 48 hours. In Panel B, a flow cytometry plot is shown demonstrating the expression of CD99 (expressed, but at low levels) in iPSC. Panel C is also a flow cytometry plot showing the high expression of CD99 in DIPG tumors.

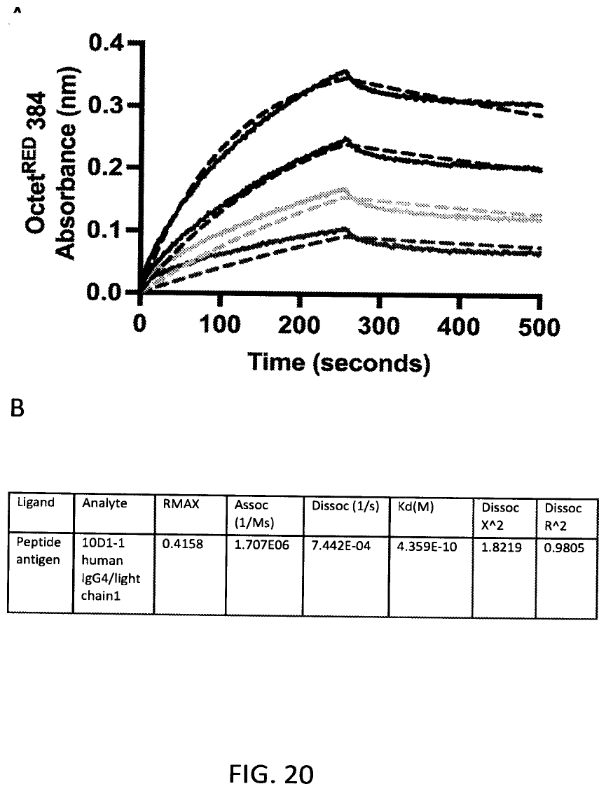

The affinity of one embodiment of the disclosed anti-CD99 antibody was tested against the CD99 epitope. Specifically, the epitope was a peptide antigen and the anti-CD99 antibody was the embodiment of 10D1-1 human IgG4/light chain1. As shown in FIG. 20. affinity was analyzed using an SA biosensor, and association and dissociation constants were monitored using OctetRED 384. These experiments indicate that the association rate is about 1.707E06 per Ms⁻¹, the dissociation rate is 7.442E-04 s⁻¹, and the dissociation rate constant, Kd, is 4.359E-10 M or about 436 pM. In various embodiments, the affinity of the disclosed antibodies may be greater than about 1 nM, 900 pM, 850 pM, 800 pM, 750 pM, 700 pM, 650 pM, 600 pM, 550 pM, 500 pM, 450 pM. 400 pM, 350 pM, 300 pM, 250 pM, 200 pM, 150 pM, 100 pM, 90 pM, 80 pM, 70 pM. 60 pM, 50 pM, 40 pM, 30 pM, 20 pM, or 10 pM, and less than about 1 pM, 10 pM, 50 pM, 100 pM, 200 pM, 300 pM, 400 pM, 500 pM, 600 pM. 700 pM, 800 pM, or900 pM. In many embodiments, the Kd is is greater than 600 pM and less than 300 pM, for example, between 550 pM and 350 pM, or between 500 pM and 400 pM as determined using the disclosed epitope and SA biosensor analysis.

Disclosed herein are various antibodies with affinity for the surface marker CD99. In many embodiments, the antibodies may comprise one or more CDRs, for example, a Heavy Chain CDR1 of SEQ ID NO: 6, a Heavy Chain CDR2 of SEQ ID NO: 7, a Heavy Chain CDR3 of SEQ ID NO: 8, a Light Chain CDR1 of SEQ ID NO: 10, a Light Chain CDR2 of SEQ ID NO: 11, and/or a Light Chain CDR3 of SEQ ID NO: 12. In some embodiments; the antibody may comprise a Variable Light Chain at least 80% identical to SEQ ID NO: 2 and/or a Variable Heavy Chain at least 80% identical to SEQ ID NO: 3. In various embodiments, the antibody may comprise an Fc region. for example, an Fc region of IgG4 with at least 80% identical to SEQ ID NO: 4. The disclosed antibodies may be useful for the treatment of one or more of DIPG, Ewing Sarcoma, acute myeloid leukemia (AML), ependymoma, and neuroblastoma. In many embodiments, the disclosed antibody may have an affinity greater than about 700 pM for an epitope of CD99. Also disclosed are therapeutic compositions comprising the claimed antibodies. Also disclosed are methods of inducing apoptosis in a cancer cell and methods of reducing or inhibiting growth of a cancer cell, the methods comprising contacting a CD99 protein on a surface of the cancer cell with the presently disclosed antibodies

Also disclosed are methods of treating DIPG in a subject in need thereof, comprising administering to the subject a therapeutic amount of an antibody having an affinity for an epitope of CD99. In some embodiments, the target epitope may have a sequence at least 80% identical to SEQ ID NO: 1, maybe an IgG4 antibody, may be chimeric or humanized. and/or may have an affinity for CD99 greater than about 700 pM. In many embodiments, the antibody may comprise one or more CDR, for example, a Heavy Chain CDR1 of SEQ ID NO: 6, a Heavy Chain CDR2 of SEQ ID NO: 7, a Heavy Chain CDR3 of SEQ ID NO: 8, a Light Chain CDR1 of SEQ ID NO: 10, a Light Chain CDR2 of SEQ ID NO: 11, and a Light Chain CDR3 of SEQ ID NO: 12. In some embodiments, the antibody may comprise a Variable Light Chain at least 80% identical to SEQ ID NO: 2 and/or a Variable Heavy Chain at least 80% identical to SEQ ID NO: 3.

Also disclosed are methods of treating a solid tumor, comprising directing radiation energy toward the solid tumor: and contacting at least one cell within the solid tumor with an anti-CD99 antibody. In some embodiments, the antibody may be chimeric or humanized. In some embodiments, the antibody of the methods may comprise a CDR, for example, a Heavy Chain CDR1 of SEQ ID NO: 6, a Heavy Chain CDR2 of SEQ ID NO: 7, a Heavy Chain CDR3 of SEQ ID NO: 8, a Light Chain CDR1 of SEQ ID NO: 10, a Light Chain CDR2 of SEQ ID NO: 11, and a Light Chain CDR3 of SEQ ID NO: 12. In some embodiments, the antibody may comprise a Variable Light Chain at least 80% identical to SEQ ID NO: 2 and/or a Variable Heavy Chain at least 80% identical to SEQ ID NO: 3. In some embodiments, the disclosed methods may be useful in treating one or more of acute myeloid leukemia (AML), ependymoma, neuroblastoma, Ewing Sarcoma, or diffuse intrinsic pontine glioma (DIPG), and the antibody binds a target epitope of CD99 with an affinity greater than about 700 pM.

Also disclosed are methods of detecting a cancerous cell. comprising exposing a potentially cancerous cell to an anti-CD99 antibody, allowing the anti-CD99 antibody to bind the potentially cancerous cell, and analyzing the number of anti-CD99 on a surface of the potentially cancerous cell. In some embodiments, the antibody may be chimeric or humanized. In many embodiments. the antibody may comprise one or more CDR sequences, for example, one or more of a Heavy Chain CDR1 of SEQ ID NO: 6, a Heavy Chain CDR2 of SEQ ID NO: 7, a Heavy Chain CDR3 of SEQ ID NO: 8, a Light Chain CDR1 of SEQ ID NO: 10, a Light Chain CDR2 of SEQ ID NO: 11, and a Light Chain CDR3 of SEQ ID NO: 12. In some embodiments, the antibody may comprise a Variable Light Chain at least 80% identical to SEQ ID NO: 2 and/or a Variable Heavy Chain at least 80% identical to SEQ ID NO: 3. In some embodiments, the disclosed methods may be useful in treating cancerous cells associated with acute myeloid leukemia (AML), ependymoma, neuroblastoma, Ewing Sarcoma, or diffuse intrinsic pontine glioma (DIPG). and the antibody binds a target epitope of CD99 with an affinity greater than about 700 pM.

The antibody disclosed herein may not alter proliferation or induce cell death when bound to non-cancerous cells.

### Definitions

As used herein. the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

An amino acid within any of the presently claimed proteins, peptides, chains, immunoglobulins. and antibodies may be substituted without extending beyond the claimed molecule. The amino acid (aa or a.a.) residue can be replaced by a residue having similar physiochemical characteristics, that is a ‘conservative substitution’—e.g., substituting one aliphatic residue for another (such as Ile, Val, Leu, or Ala for one another), or substitution of one polar residue for another (such as between Lys and Arg: Glu and Asp; or Gln and Asn). Other such conservative substitutions, for example based on size, charge, polarity, hydrophobicity, chain rigidity/orientation, etc., are well known in the art of protein engineering. Polypeptides comprising conservative amino acid substitutions can be tested in any one of the assays described herein to confirm that a desired activity, e.g. binding, specificity, and/or function of a native or reference polypeptide is achieved.

“Amino acid identity,” “residue identity,” “identity,” and the like, as used herein refers to the structure of the functional group (R group) on the poly peptide backbone at a given position. Naturally occurring amino acid identities are (name/3-letter code/one-letter code): alanine/ala/A; arginine/arg/R; asparagine/asn/N; aspartic acid/asp/D; cysteine/cys/C; glutamine/gln/Q; glutamic acid/glu/E; glycine/gly/G; histidine/his/H; isoleucine/ile/I; leucine/leu/L; lysine/lys/K; methionine/met/M; phenylalanine/phe/F; proline/pro/P; serine/ser/S; threonine/thr/T; tryptophan/trp/W; tyrosine/tyr/Y; and valine/val/V.

Similarity between amino acid or peptide sequences is expressed in terms of the similarity between two sequences, otherwise referred to as sequence identity. Sequence identity is frequently measured in terms of percentage identity (percentage of identical residues for peptides or bases for nucleic acids: or similarity or homology): the higher the percentage. the more similar the two sequences are. Complete identity is 100% identical over a given sequence, for example 50, 100, 150, or 200 bases or residues.

Amino acid or nucleic acid sequences can be substituted to be at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to a native or reference sequence (i.e. one or more of the disclosed sequences). The degree of homology (percent identity) between a native and variant sequence can be determined, for example, by comparing the two sequences using freely available computer programs commonly employed for this purpose on the world wide web (e.g., BLASTp or BLASTn with default settings). In the case of protein substitutions, amino acid deletions or insertions may also be included.

Amino acids can be grouped according to similarities in the properties of their side chains (in A. L. Lehninger, in Biochemistry. second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: leucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and understood by those of skill in the art.

“Nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect. the nucleic acid can be DNA. In another aspect, the nucleic acid can be RNA. Suitable DNA can include. e.g., genomic DNA, cDNA, or vector DNA. Suitable RNA can include, e.g., mRNA.

“Expression” as used herein, refers to cellular processes involved in producing, displaying (e.g., on or at a cell's surface/outer membrane), or secreting RNA and proteins including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

The terms antibody and immunoglobulin may be used to refer to a tetrameric glycoprotein that consists of two heavy chains and two light chains, each comprising a variable region and a constant region. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. The terms heavy chain and light chain may refer to substantially full-length canonical immunoglobulin light and heavy chains (see e.g., Immunobiology, 5th Edition, Janeway and Travers et al., Eds., 2001). Antigen-binding portions thereof may be produced by natural methods, recombinant DNA techniques, or by enzymatic or chemical cleavage of intact antibodies. The term antibody includes monoclonal antibodies, polyclonal antibodies, chimeric antibodies, human antibodies, and humanized antibodies.

As used herein, the term “specific binding” refers to the ability of a molecule to preferentially bind to a particular epitope with more avidity than to other, related epitopes, for example where the epitope and related epitopes are found on a single protein. In some instances, a specific binding interaction may discriminate between epitopes with a specificity of 10-fold or more. such as 100-fold or more, or 1000-fold or more.

As used herein, the terms “affinity” and “avidity” have the same meaning and may be used interchangeably herein. “Affinity” refers to the strength of binding, with increased binding affinity being correlated with a lower Kd.

The term epitope refers to a binding determinant, which is specifically bound/identified by an antibody or immunoglobulin, as defined above. The antibody or immunoglobulin may specifically bind to/interact with conformational or continuous epitopes, which are unique for a target structure, e.g. the CD99 surface protein. A continuous or linear epitope consists of two or more discrete amino acid residues, which are present in a single linear segment of a polypeptide chain. A conformational or discontinuous epitope is characterized for polypeptide antigens by the presence of two or more discrete amino acid residues which are separated in the primary sequence, but come together on the surface of the molecule when the polypeptide folds into the native protein/antigen.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “cancer” “cancerous” means a disease, condition, or group of diseases involving abnormal cell growth. In many embodiments cancer cells or cancerous cells may possess the potential to invade or spread to other tissues, organs, and parts of the body. Cancer may refer to blood and solid tissue cancers, for example acute myeloid leukemia (AML), ependymoma, neuroblastoma, Ewing Sarcoma, or diffuse intrinsic pontine glioma (DIPG).

The term “effective amount” refers to an amount of a compound of the invention or other active ingredient sufficient to provide a therapeutic or prophylactic benefit in the treatment or prevention of a disease or to delay or minimize symptoms associated with a disease. Further, a therapeutically effective amount with respect to a compound of the invention means the amount of therapeutic agent alone. or in combination with other therapies, that provides a therapeutic benefit in the treatment or prevention of disease. Used in connection with a compound of the invention. the term can encompass an amount that improves overall therapy, reduces or avoids symptoms or causes of disease. or enhances the therapeutic efficacy or synergies with another therapeutic agent.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size: inhibit (i.e., slow to some extent and preferably stop) cancer cell infiltration into peripheral organs; inhibit (i.e., slow to some extent and preferably stop) tumor metastasis: inhibit, to some extent, tumor growth, tumor cell division or growth: cause cancer cell death; and/or relieve to some extent one or more of the symptoms associated with cancer. To the extent the therapeutic may prevent growth and/or kill existing cancer cells. it may be cytostatic and/or cytotoxic. For cancer therapy. efficacy can be measured, for example, by assessing the time to disease progression (TTP) and/or determining the response rate (RR).

Humanized forms of non-human (e.g., rodent) antibodies are chimeric antibodies that contain minimal sequence derived from non-human immunoglobulin. In some cases, humanized antibodies are immunoglobulins in which residues from a hypervariable region of the recipient are replaced by residues from a hypervariable region of a non-human species (donor antibody) such as a mouse, rat, rabbit or nonhuman primate having the desired specificity, affinity, and capacity. In some instances, framework region (FR) residues of a non-human immunoglobulin may be replaced by corresponding human residues or vice versa. Furthermore, humanized antibodies may comprise residues that are not found in the recipient antibody or in the donor antibody. These modifications are made to further refine antibody performance. In general, the humanized antibody will comprise substantially all of at least one, and typically two, variable domains or CDRs of those domains in which all or substantially all of the hypervariable loops correspond to those of a non-human immunoglobulin and all or substantially all of the FRs are those of a human immunoglobulin sequence. A humanized antibody optionally also will comprise at least a portion of an immunoglobulin constant region (Fc), typically that of a human immunoglobulin. For further details, see Jones et al., Nature 321:522-525 (1986): Riechmann et al., Nature 332:323-329 (1988): and Presta, Curr. Op. Struct. Biol. 2:593-596 (1992).

A “patient’ or “subject” refers to an animal, such as a human, cow, horse, sheep, lamb, pig, chicken, turkey, quail, cat, dog, mouse, rat, rabbit or guinea pig. The animal can be a mammal such as a non-primate and a primate (e.g., monkey and human). In one embodiment, a patient is a human, such as a human infant, child, adolescent, or adult.

A “subject in need,” “patient in need,” or those “in need of treatment” include patients or subjects with existing disease (e.g. cancer), as well as those at risk of the disease. The terms also include human and other mammalian subjects that receive either prophylactic or therapeutic treatments as disclosed herein.

The terms “treat,” “treating,” and “treatment” refer to eliminating, reducing, suppressing, or ameliorating, either temporarily or permanently, partially or completely, a clinical symptom, manifestation, or progression of an event, disease or condition associated with the intervertebral disc disorders and diseases described herein. As is recognized in the pertinent field, methods, and therapeutics employed as therapies may reduce the severity of a given disease state but need not abolish every manifestation of the disease to be regarded as useful. Similarly, a prophylactically administered treatment need not be completely effective in preventing the onset of a condition to constitute a viable prophylactic method or agent. Simply reducing the impact of a disease (for example, as disclosed herein, increasing intervertebral disc height, reducing back pain, etc., and/or reducing the number or severity of associated symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient one or more therapeutic treatments in an amount, duration, and repetition sufficient to induce a sustained improvement over pre-existing conditions or a baseline indicator that reflects the severity of the particular disorder.

### EXAMPLES

### Methods

### Antibody Amino Acid Sequence

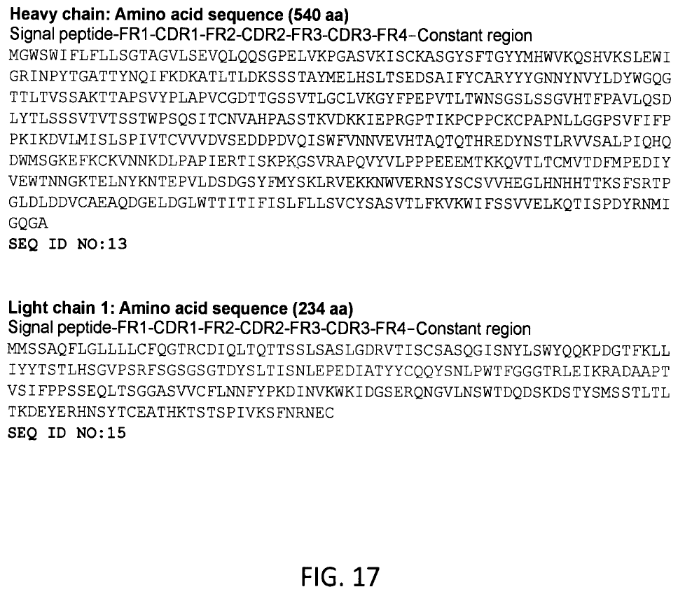

FIG. 17 presents the amino acid sequence of the variable heavy (A: top) and light (B: bottom) regions of one embodiment of the disclosed anti-CD99 antibody. CDRs are marked as indicated.

XCELLigence cell proliferation assay: Cells (˜2×10⁵ cells/plate), were seeded on a gold-plated 96-well E-plate, and the cell growth index was measured in real-time using xCELLigence. 24 hrs after seeding, cells were treated with either the control IgG4 or with 10D1 antibody at different doses, and the change in the cell growth index was measured over time.

Incucyte apoptosis assay: Cells (˜2×10⁶ cells/well) were cultured in a flat-bottom 6-well plate on day 0. On day 2, the cells were treated with 10D1 CD99 antibody or with IgG4 controls at different concentrations. On day 3, the CellEvent Caspase3/7 Green Detection Reagent (Invitrogen) was added to each well at a final concentration of 2 mM. The apoptosis induced after treatments were measured using a Incucyte S3 Live Cell Analysis System. The increase in apoptosis over time was measured by an increase in the green fluorescence, which is a measure of the amount of dye cleaved by the activated caspase3/7. The number of green objects was counted and expressed as Total Green Object Integrated Intensity (GCU×mm²/well) which was normalized to the percentage cells confluence, i.e., phase confluence, calculated from the phase contrast image of that well at the corresponding time point. The change in the normalized intensity was plotted against time.

In combination with radiation: Cells were seeded as described above on day 0. On day 1 cells were subjected to indicated varying doses of radiation. On day 2, cells were treated with varying concentrations of either control IgG4 or 10D1 CD99 antibody. On day 3, caspase3/7 detection reagents were added and the experiment was performed as described above.

Flow cytometry assay: The cell surface expression of CD99 was determined using 10D1 CD99 antibody by flow cytometry. Briefly, cells were resuspended in FACS buffer, cells are fixed with 70% ethanol and stained with 10D1 CD99 primary antibody for 30 minutes. Stained cells were then washed and CD99 positive cells were detected using PE-conjugated anti-human IgG Fc secondary antibody. DAPI staining was used to exclude dead cells.

The binding of 10D1 CD99 antibody was determined by treating DIPG cells first with the 10D1 antibody, followed by fixing the cells as described earlier for flow measurement of CD99 using the commercially available 0662 CD99 antibody. Flow cytometric measurements were performed using Cytoflex flow instrument and data analysis was done using FlowJO software. Results were expressed as mean fluorescent intensity fold change to isotype-matched control Ig staining or secondary antibody staining.

### Immunohistochemistry (IHC)

Normal and patient tumors or mouse xenograft tissues were fixed in formalin, paraffin-embedded and sectioned. Stained for CD99 using 10D1 CD99 antibody. The staining of 10D1 antibody was compared with other commercial CD99 antibodies (Sigma Aldrich. Thermo fisher. Atlas. Millipore). IHC was performed at the pathology core facility at the University of Colorado Denver (UCD). The pictures of the antibody-stained sections were taken using an Olympus BX40 microscope with a 40× objective lens.

### In Vivo Assays: DIPG Mouse Models

a. Human DIPG cells tagged with luciferase-GFP (BT245-Luc2-GFP or SU-DIPGXIII *-Luc2-GFP) were implanted in the pons of 6- to 8-week-old female nude mice. Briefly, a suspension of ˜1×10⁵ cells in 2 μl serum-free media were stereotactically injected at a rate of 500 nL/min into the brain at a site 0.8 mm lateral to midline, 0.5 mm posterior to lambda, and 5.00 mm ventral to the surface of the skull. Tumor formation was monitored by bioluminescent imaging (BLI) once per week using Xenogen IVIS 200 imaging machine. After conformation of tumor establishment in the pons, with BLI corresponding to ˜10⁵ to 10⁶ photons using IVIS, animals received treatment either by i.v. (tail vein) or by loco-regional delivery.
b. Tail vein (i.v.) delivery of the antibody: After tumor establishment, mice were randomized into 2 treatment groups as follows: (1) IgG4 (control), n=8-10 and (2) 10D1 CD99 n=8-10 (8 mg/Kg/day, administered intravenously every other day for a total of 8 infusions.
c. Loco-regional delivery of the antibody:
i. Antibody administered at the 4^{th} ventricle: After tumor establishment, mice were randomized into 2 treatment groups as follows: (1) IgG4 (control, 0.8 mg/Kg/day)) and (2) 10D1 CD99 (0.8 mg/Kg/day), a single dose was administered in the 4^{th} ventricle.
ii. Antibody administered directly to the tumor site, pons (Convection Enhanced Delivery. CED): After tumor establishment, mice were randomized into 2 treatment groups as follows: (1) IgG4 (control) and (2) 10D1 CD99 (0.8 mg/Kg/day),a single dose was administered to the pons tumor target site.

### In Vivo Assays: Ewing Sarcoma Mouse Xenograft Models

About 7 million luciferase-expressing ES cells, A4573 cells, were implanted in the pretibial space [18]. These ES cell line xenografts established with implantation of 7 million cells will result in a palpable tumor with BLI of greater than 10⁶ photons within 14-18 days after injection. Once the tumor is palpable and the tumor establishment is confirmed by IVIS bioluminescent imaging (BLI), the animals will be randomized into two groups. Groups 1 and 2 received 3 doses of either IgG4 or 10D1-CD99 antibody (16 mg/kg/day), intravenously (I.V.) on alternative days.

In both models, tumor growth and response to therapy were determined biweekly by BLI imaging. The tumor take rate was 100%. Body weight was measured once a week and mice were monitored daily and those reaching end-point were euthanized according to IACUC protocols by CO₂ asphyxiation, when they show signs of either neurological deficit, failure to ambulate, body score less than 2, or weight loss greater than 20%. Animal survival was plotted to measure the increase in survival with the treatment.

### Western Blot for Protein Detection

Protein analysis was done with whole cell lysates isolated from DIPG patient tumors and cell lines with indicated treatments. The protein lysates were collected in RIPA buffer supplemented with protease inhibitor cocktail tablets, sodium vanadate, and sodium molybdate as previously described [19]. After the protein concentrations were determined using BCA Assay, western blotting was performed.

### REFERENCES

Curtin SC, Minino AM, Anderson RN: Declines in Cancer Death Rates Among Children and Adolescents in the United States, 1999-2014. NCHS Data Brief 2016 (257): 1-8.
Grimm SA. Chamberlain MC: Brainstem glioma: a review. Curr Neurol Neurosci Rep 2013, 13 (5): 346.
Bartels U, Hawkins C, Vezina G. Kun L, Souweidane M, Bouffet E: Proceedings of the Diffuse intrinsic pontine glioma (DIPG) Toronto Think Tank: advancing basic and translational research and cooperation in DIPG. Journal of neuro-oncology 2011, 105 (1): 119-125.
Jansen MH, van Vuurden DG, Vandertop WP, Kaspers GJ: Diffuse intrinsic pontine gliomas: a systematic update on clinical trials and biology. Cancer treatment reviews 2012, 38 (1): 27-35.
Warren KE: Diffuse intrinsic pontine glioma: poised for progress. Frontiers in oncology 2012, 2:205.
Buczkowicz P, Hawkins C: Pathology, Molecular Genetics, and Epigenetics of Diffuse Intrinsic Pontine Glioma. Frontiers in oncology 2015, 5:147.
Lewis PW, Muller MM. Koletsky MS, Cordero F, Lin S, Banaszynski LA, Garcia BA, Muir TW, Becher OJ, Allis CD: Inhibition of PRC2 activity by a gain-of-function H3 mutation found in pediatric glioblastoma. Science 2013, 340(6134):857-861.
Warren KE, Killian K, Suuriniemi M, Wang Y, Quezado M, Meltzer PS: Genomic aberrations in pediatric Diffuse intrinsic pontine gliomas. Neuro-oncology 2012, 14(3):326-332.
Chan KM, Fang D, Gan H, Hashizume R, Yu C, Schroeder M, Gupta N, Mueller S, James CD, Jenkins R et al: The histone H3.3K27M mutation in pediatric glioma reprograms H3K27 methylation and gene expression. Genes Dev 2013, 27(9):985-990.
Halvorson KG. Barton KL. Schroeder K. Misuraca KL. Hoeman C. Chung A. Crabtree DM, Cordero FJ, Singh R, Spasojevic I et al: A high-throughput in vitro drug screen in a genetically engineered mouse model of Diffuse intrinsic pontine glioma identifies BMS-754807 as a promising therapeutic agent. PLoS One 2015, 10(3): e0118926.
Pasello M, Manara MC, Scotlandi K: CD99 at the crossroads of physiology and pathology. J Cell Commun Signal 2018, 12(1):55-68.
Pedersen H, Schmiegelow K, Hamerlik P: Radio-Resistance and DNA Repair in Pediatric Diffuse Midline Gliomas. Cancers (Basel) 2020, 12(10).
Balamuth NJ, Womer RB: Ewing's sarcoma. Lancet Oncol 2010, 11(2):184-192.
Bleyer A, Barr R, Hayes-Lattin B, Thomas D, Ellis C, Anderson B, Biology, Clinical Trials Subgroups of the USNCIPRGIA, Young Adult O: The distinctive biology of cancer in adolescents and young adults. Nat Rev Cancer 2008, 8(4):288-298.
Scotlandi K, Baldini N, Cerisano V, Manara MC, Benini S, Serra M, Lollini PL, Nanni P. Nicoletti G. Bernard G et al: CD99 engagement: an effective therapeutic strategy for Ewing tumors. Cancer Res 2000, 60(18):5134-5142.
Benini S. Gamberi G, Cocchi S, Garbetta J, Alberti L, Righi A, Gambarotti M, Picci P, Ferrari S: Detection of circulating tumor cells in liquid biopsy from Ewing sarcoma patients. Cancer Manag Res 2018, 10:49-60.
Panditharatna E, Kilburn LB, Aboian MS, Kambhampati M, Gordish-Dressman H, Magge SN. Gupta N, Myseros JS, Hwang EI, Kline C et al: Clinically Relevant and Minimally Invasive Tumor Surveillance of Pediatric Diffuse Midline Gliomas Using Patient-Derived Liquid Biopsy. Clin Cancer Res 2018, 24(23):5850-5859.
Goldstein SD. Hayashi M. Albert CM. Jackson KW. Loeb DM: An orthotopic xenograft model with survival hindlimb amputation allows investigation of the effect of tumor microenvironment on sarcoma metastasis. Clin Exp Metastasis 2015, 32(7):703-715.
Balakrishnan I, Danis E, Pierce A, Madhavan K, Wang D, Dahl N, Sanford B, Birks DK, Davidson N, Metselaar DS et al: Senescence Induced by BMIl Inhibition Is a Therapeutic Vulnerability in H3K27M-Mutant DIPG. Cell Rep 2020, 33(3):108286.

What is claimed is:

1. An antibody directed to CD99, comprising:
a Heavy Chain CDR1 of SEQ ID NO: 6;
a Heavy Chain CDR2 of SEQ ID NO: 7;
a Heavy Chain CDR3 of SEQ ID NO: 8;
a Light Chain CDR1 of SEQ ID NO: 10;
a Light Chain CDR2 of SEQ ID NO: 11; and
a Light Chain CDR3 of SEQ ID NO: 12.

2. The antibody directed to CD99 of claim 1, comprising a Variable Light Chain at least 80% identical to SEQ ID NO: 2 and/or a Variable Heavy Chain at least 80% identical to SEQ ID NO: 3.

3. The antibody directed to CD99 of any one of claim 1 or 2, comprising an IgG4 Fc at least 80% identical to SEQ ID NO: 4.

4. The antibody directed to CD99 of any one of any one of claims 1 to 3, for treatment of DIPG, Ewing Sarcoma, acute myeloid leukemia (AML), ependymoma, or neuroblastoma.

5. A therapeutic composition comprising any one of the antibodies directed to CD99 of any one of claims 1 to 4, wherein the antibody binds a target epitope of CD99 with an affinity greater than about 700 pM.

6. A method of inducing apoptosis in a cancer cell, comprising:
contacting a CD99 protein on a surface of the cancer cell with an antibody directed to CD99 of any one of claims 1 to 4.

7. A method of reducing or inhibiting growth of a cancer cell, comprising:
contacting a CD99 protein on a surface of the cancer cell with an antibody directed to CD99 of any one of claims 1 to 4.

8. A method of treating DIPG in a subject in need thereof, comprising:
administering to the subject a therapeutic amount of an antibody having affinity for an epitope of CD99.

9. The method of claim 8, wherein the epitope of CD99 is a peptide at least 80% identical to SEQ ID NO: 1.

10. The method of claims 8 to 9, wherein the antibody is an IgG4 antibody.

11. The method of any one of any one of claims 8 to 10, wherein the antibody is chimeric or humanized and/or wherein the antibody binds a target epitope of CD99 with an affinity greater than about 700 pM.

12. The method of any one of any one of claims 8 to 11, wherein the antibody comprises;
a Heavy Chain CDR1 of SEQ ID NO: 6;
a Heavy Chain CDR2 of SEQ ID NO: 7;
a Heavy Chain CDR3 of SEQ ID NO: 8;
a Light Chain CDR1 of SEQ ID NO: 10;
a Light Chain CDR2 of SEQ ID NO: 11; and
a Light Chain CDR3 of SEQ ID NO: 12.

13. The method of any one of any one of claims 8 to 12, wherein the antibody comprises; a Variable Light Chain at least 80% identical to SEQ ID NO: 2 and/or a Variable Heavy Chain at least 80% identical to SEQ ID NO: 3.

14. A method of treating a solid tumor, comprising:
directing radiation energy toward the solid tumor; and
contacting at least one cell within the solid tumor with an anti-CD99 antibody, thereby, treating the solid tumor.

15. The method of claim 14, wherein the antibody is chimeric or humanized.

16. The method of claim 14 or 15, wherein the antibody comprises;
a Heavy Chain CDR1 of SEQ ID NO: 6;
a Heavy Chain CDR2 of SEQ ID NO: 7;
a Heavy Chain CDR3 of SEQ ID NO: 8;
a Light Chain CDR1 of SEQ ID NO: 10;
a Light Chain CDR2 of SEQ ID NO: 11; and
a Light Chain CDR3 of SEQ ID NO: 12.

17. The method of any one of claims 14 to 16, wherein the antibody comprises: a Variable Light Chain at least 80% identical to SEQ ID NO: 2 and/or a Variable Heavy Chain at least 80% identical to SEQ ID NO: 3.

18. A method of detecting a cancerous cell, comprising:
exposing a potentially cancerous cell to an anti-CD99 antibody:
allowing the anti-CD99 antibody to bind the potentially cancerous cell:
analyzing the number of anti-CD99 on a surface of the potentially cancerous cell.

19. The method of claim 18, wherein the anti-CD99 antibody is chimeric or humanized.

20. The method of claim 18 or 19, wherein the anti-CD99 antibody comprises:
a Heavy Chain CDR1 of SEQ ID NO: 6;
a Heavy Chain CDR2 of SEQ ID NO: 7;
a Heavy Chain CDR3 of SEQ ID NO: 8;
a Light Chain CDR1 of SEQ ID NO: 10;
a Light Chain CDR2 of SEQ ID NO: 11; and
a Light Chain CDR3 of SEQ ID NO: 12.

21. The method of any one of claims 18 to 20, wherein the anti-CD99 antibody comprises: a Variable Light Chain at least 80% identical to SEQ ID NO: 2 and/or a Variable Heavy Chain at least 80% identical to SEQ ID NO: 3.

23. The method of any one of claims 14-17, wherein the tumor or cells are associated with acute myeloid leukemia (AML), ependymoma, neuroblastoma, Ewing Sarcoma, or diffuse intrinsic pontine glioma (DIPG), and the antibody binds a target epitope of CD99 with an affinity greater than about 700 pM.

24. The method of any one of claims 18-21, wherein the potentially cancerous cell is associated with acute myeloid leukemia (AML), ependymoma, neuroblastoma, Ewing Sarcoma, or diffuse intrinsic pontine glioma (DIPG), and the antibody binds a target epitope of CD99 with an affinity greater than about 700 pM.

25. The antibody, therapeutic composition, or method of any one of claims 1-24, wherein the antibody does not alter proliferation or induce cell death when bound to non-cancerous cells.
